(19) United States
(12) Patent Application Publication
(10) Pub. No.: US 2022/0068492 A1
(43) Pub. Date: Mar. 3, 2022

(54) SYSTEM AND METHOD FOR SELECTING REQUIRED PARAMETERS FOR PREDICTING OR DETECTING A MEDICAL CONDITION OF A PATIENT

(71) Applicant: GYNISUS INC., Los Angeles, CA (US)

(72) Inventor: Hila FRIEDMANN, Bat Yam (IL)

(21) Appl. No.: 17/422,896

(22) PCT Filed: Jan. 14, 2020
(86) PCT No.: PCT/IL2020/050059

§ 371 Date: Jul. 14, 2021

Related U.S. Application Data
(60) Provisional application No. 62/894,910, filed on Sep. 2, 2019.
(60) Provisional application No. 62/872,803, filed on Jul. 11, 2019.
(60) Provisional application No. 62/791,914, filed on Jan. 14, 2019.

(51) Int. Cl. G16H 50/30; G16H 50/70; G16H 50/20; G16H 10/60

(52) U.S. Cl. CPC G16H 50/30; G16H 10/60; G16H 50/20; G16H 50/70

(57) ABSTRACT

A system and a method of selecting required parameters for prediction or detection of a medical condition are disclosed. The method may include: receiving sparse data pertaining at least to electronic medical records (EMR) of at least one patient; preprocessing the sparse data; completing the sparse data by adding at least a portion of a missing data using a cross-validation process; and selecting the required parameters from the completed data

### CROSS REFERENCE TO RELATED APPLICATIONS

This application claiming the benefit of United States Provisional Patent Application No. 62/791,914, filed Jan. 14, 2019, and U.S. Provisional Patent Application No. 62/872,803, filed Jul. 11, 2019 and U.S. Provisional Patent Application No. 62/894,910, filed Sep. 3, 2019 all of which are hereby incorporated by reference.

### FIELD OF THE INVENTION

The present invention relates generally to the field of healthcare. More specifically, the present invention relates to the field of determining required parameters for predicting or detecting a medical condition of a patient.

### BACKGROUND OF THE INVENTION

Electronic medical records (EMR) or electronic health records (EHR) are systematized collections of data pertaining to patient and population, including electronically stored health information in a digital format. These records include data collected over a long period of time, and associated with specific patients collected from one or more health care settings, such as, doctors, hospitals, laboratories, diagnostic imaging centers and the like.

There have being many attempts to use EMRs as a diagnostic tool, using machine learning processes. For example, such a tool may allow to train a machine learning (ML) model to diagnose a medical condition (e.g., an illness) in a person, using data collected from diagnosed people.

One of the major challenges in using EMR is the extreme sparsity of the data. This data includes thousands of different parameters, of which only some of them may be included in a single person's EMR. Therefore, the parameters (e.g., laboratory tests, diagnosis, radiography images, MRI images, and the like) included in each file for each person are normally very different and sparse. Therefore, even for two people having some similarities (e.g., age, gender, at least one diagnosed illness, etc.) the chance of having similar EMR data is very low.

Accordingly, any attempt to use such the EMR may include methods of overcoming the sparse nature of the data.

Furthermore, never before an attempt was made to use such a sparse data to predict a future medical condition in a generally healthy person. The term “future” may be used herein to indicate that the relevant person may not be suffering from any known or common symptoms indicative of that medical condition. Specifically, no attempt has been made to predict a pregnancy related medical condition (e.g., gestational diabetes mullites (GDM)) in a woman who is not pregnant.

Accordingly, a system and method according to embodiments of the invention may allow predict a medical precondition in a patient not yet showing any known or common symptoms, before the appearance of the medical condition based on EMR.

### SUMMARY OF THE INVENTION

Some aspects of the invention may be related to a method of selecting, by at least one processor, required parameters for prediction or detection of a medical condition. In some embodiments, the method may include: receiving sparse data pertaining at least to electronic medical records (EMR) of at least one patient; preprocessing the sparse data; completing the sparse data by adding at least a portion of a missing data using a cross-validation process; and selecting the required parameters from the completed data.

In some embodiments, selecting the required parameters is by a module adapted to: receiving sparse data pertaining to at least EMR of a plurality of patients; preprocessing the sparse data; completing the sparse data, pertaining to EMR of a plurality of patients, by adding at least a portion of a missing data using the cross-validation process; and arranging the parameters in the completed data according to their level of importance. In some embodiments, inclusion of data received only from patients diagnosed with the medical condition. In some embodiments, the preprocessing of the sparse data may include inclusion of the data received from patient in a precondition

In some embodiments, the level of importance is determined by feature importance method. In some embodiments, the level of importance is determined by information gain calculations may include: receiving for each parameter in the sparse data: a first number of patients included in a first group of patients to which the EMR data comprises the parameter; and a second number of patients included in a second group of patients diagnosed with the condition, the second group is selected from the first group.

In some embodiments, the method may further include: identifying in the required parameters, one or more parameters related to medical tests; and determining a list of required medical tests for prediction or detection of a medical condition based on the identified parameters. In some embodiments, receiving the sparse data pertaining to the EMR is for a group of patients belonging to at least one category; and wherein the required parameters are selected to best fit the group of patients.

In some embodiments, the method may further include: training a machine learning (ML) module to predict or detect the medical condition based on the completed data and the level of importance. In some embodiments, the method may further include: predicting or detecting, for the at least one patient, by the trained ML module, a future appearance of the medical condition based on the completed data.

In some embodiments, the method may further include: receiving a set of medical conditions; predicting for the at the least one patient a probability for future appearance of each medical condition in the set; and determining a risk level for the at the least one patient based on the predicted probability of each medical condition in the set. In some embodiments, completing the sparse data may include: completing parameters missing from the sparse data with parameters having a sufficient similarity, and wherein the similarity is determined based on at least one of: a similarity between patients, similarity between parameters and a combination thereof.

In some embodiments, the method may further include: determining a first reliability level of each parameter based on a time associated with the parameter. In some embodiments, the method may further include: determining a second reliability level of each parameter based on number of occurrences of each parameter for different patients.

In some embodiments, the method may further include: determining a physician profile for a plurality of physicians; determining for each physician profile a decision diversity function in identical medical situation; and correcting parameters related to the physician's inputs based on the determined decision diversity function.

In some embodiments, preprocessing of the sparse data may include: normalizing time dependent parameters in the sparse data received from different patients to a single timeline. In some embodiments, the method may further include: dividing the timeline into time intervals; associating each time dependent parameter with a specific time interval; determining a decay rate parameter for each time interval; and calculating a weight of each time dependent parameter using the corresponding decay rate parameter.

In some embodiments, at least one parameter from the sparse data is a monitored parameter, and the method may further include: detecting at least one abnormality in the monitored parameter; and assigning a representing value to the detected at least one abnormality. In some embodiments, the preprocessing of the sparse data may include: identifying category dependent parameters in the sparse data; and assigning a score for each category. In some embodiments, the at least one category is selected from: religion, ethnicity, race, spoken language, age, gender, citizenship, place of birth, and place of living, social economic level, insurance type, education, occupation. In some embodiments, the at least one category dependent parameter is one of: a genetic parameter and epigenetic parameter. In some embodiments, the at least one category dependent parameter is a geographic parameter related to a location of the patient selected from: radiation levels at the location, temperatures at the location, humidity levels at the location and altitude of the location.

In some embodiments, the method may further include: receiving additional data, from at least one of: a user device, laboratory tests and one or more sensors; preprocessing the additional data; and completing the sparse data by adding the processed additional data.

Some additional aspect of the invention may be related to a method of predicting, by at least one processor, a pregnancy medical condition in non-pregnant woman or detecting a pregnancy medical condition in a pregnant woman. In some embodiments, the method may include: receiving sparse medical data of the woman; preprocessing the sparse medical data; predicting, by a trained Machine Learning (ML) module, the probability of the woman to have the pregnancy medical condition during pregnancy.

In some embodiments, the method may further include: excluding parameters included in the medical data that were received during any of the woman previous pregnancies. In some embodiments, the pregnancy medical condition is one of: Gestational Diabetes Mellitus (GDM), preeclampsia, preterm, hypothyroidism, hyperthyroidism, cardiovascular diseases, postpartum hemorrhage, fetal heart disorder, fetal growth disorder and intra-uterine growth restriction (IUGR).

Some additional aspect of the invention may be related to a method of determining a physician profile. In some embodiments, the method may include: receiving a plurality of decisions made by the physician in response to a plurality of medical conditions, from EMR; determining a plurality of patients' profiles, based on data received from the EMR; associating each decision with a of patient's profile; and determining the physician profile based on the association.

In some embodiments, the method may further include clustering sub-group of patients from plurality of patients based on similar decisions made by the physician for a given medical condition. In some embodiments, the method may further include clustering sub-group of decisions based on similar patients' profiles. In some embodiments, determining the physician profile comprises determining, for a given medical condition and a patient's profile a predicted decision.

In some embodiments, the method may further include determining for each physician profile a diversity function in identical medical situation; and correcting parameters related to physician inputs based on the determined decision diversity function.

Some additional aspect of the invention may be related to a method of determining, by at least one processor, a starting date of pregnancy. In some embodiments, the method may include: receiving at least one of: HcG and Beta HcG measurements taken at known periods of time during pregnancies, for a plurality of women; determining real starting dates of pregnancy based on the at least one of: HcG and Beta HcG measurements for each woman in the plurality of women; finding similarity between at least one of: HcG and Beta HcG measurements and at least one other blood test, for the plurality of women; receiving the at least one other blood test for a specific woman; and determining a corrected starting date of pregnancy, for the specific woman, based on the similarity between the received at least one other blood test and the at least one of: HcG and Beta HcG measurements.

Some additional aspects of the invention may be related to a system comprising: a memory device, wherein modules of instruction code are stored, and a processor associated with the memory device, and configured to execute the modules of instruction code, whereupon execution of said modules of instruction code, the processor is further configured to perform at least one of the method steps disclosed herein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram, depicting a computing device which may be included in a system according to some embodiments of the invention;

FIG. 1B is a block diagram, depicting a system for performing at least one of: selecting required parameters for prediction or detection of a medical condition; predicting a pregnancy related medical condition in a non-pregnant woman; determining a physician profile; and determining a last menstrual period (LMP) according to some embodiments of the invention;

FIG. 2A is a block diagram, depicting a preprocessing module according to some embodiments of the invention;

FIG. 2B is a block diagram, depicting a parameterizing process in a parameterizing module according to some embodiments of the invention;

FIG. 3 is a block diagram, depicting parameters selection module according to some embodiments of the invention;

FIG. 4A is a block diagram, depicting an administrator module according to some embodiments of the invention;

FIG. 4B is an illustration of parameters hierarchy included in the administrator module, according to some embodiments of the invention;

FIG. 5A is a flowchart of a method of selecting, by at least one processor, required parameters for prediction or detection of a medical condition according to some embodiments of the invention;

FIG. 5B is a scheme showing the influence of changing a parameter on a system according to some embodiments of the invention;

FIG. 5C is a block diagram of the information analysis and flow according to some embodiments of the invention;

FIG. 6 is a flowchart of a method of selecting the required parameters according to some embodiments of the invention;

FIGS. 7A and 7B are graphs showing the correlations between blood pressure prior to pregnancy and developing gestational diabetes according to some embodiments of the invention;

FIG. 8 is a table containing correlations between vital signs tests prior to pregnancy and developing gestational diabetes according to some embodiments of the invention;

FIG. 9 is a table containing informative score per parameter of vital signs tests prior to pregnancy and the probability of developing gestational diabetes according to some embodiments of the invention;

FIG. 10 is a table containing probability and relations between demographic data (specifically Religion) prior to pregnancy and the developing of gestational diabetes according to some embodiments of the invention;

FIG. 11 is a table containing probability and relations between partial list of demographic data (specifically race) prior to pregnancy and the developing of gestational diabetes according to some embodiments of the invention;

FIG. 12 is a table containing probability and relations between partial list of demographic data (specifically race) prior to pregnancy the developing of gestational diabetes according to some embodiments of the invention;

FIG. 13 is a table containing probability and relations between partial list of demographic data (specifically language) prior to pregnancy and the developing of gestational diabetes according to some embodiments of the invention;

FIG. 14 is a table containing probability and relations between partial list of demographic data (specifically insurance type) prior to pregnancy and the developing of gestational diabetes according to some embodiments of the invention;

FIG. 15 is graph showing informative score per parameter of demographic elements prior to pregnancy and the probability of developing gestational diabetes according to some embodiments of the invention;

FIG. 16 is an illustration of information flow according to some embodiments of the invention;

FIG. 17 is a flowchart of a method of predicting a pregnancy related medical condition in non-pregnant woman according to some embodiments of the invention;

FIG. 18 is a flowchart of a method of determining a physician profile according to some embodiments of the invention;

FIG. 19 is flowchart of a method of determining a starting date of a pregnancy according to some embodiments of the invention; and

FIG. 20 is an illustration of a timeline of a woman according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, “synchronizing” or the like, may refer to operation(s) and/or process(es) of a computer, a computing platform, a computing system, or other electronic computing device (e.g., a wearable electronic computing device), that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention include a method and a system for selecting and/or determining required parameters for prediction or detection of a medical condition, for example, a future medical condition in a “healthy” person. A system and method according to embodiments of the invention may assist physicians to accurately diagnose the patient and provide the correct required treatment.

A system and method according to embodiments of the invention may allow to: complete the sparse medical data provided, for example, from EMRs, reduce the number of unnecessary medical tests, reduce strong dependency on diverse medical decisions made by doctors in similar situations and reduce a bias of medical decisions based on a specific history of each doctor.

In some embodiments, for each medical condition (e.g., an illness) and a specific person, embodiments of the system and method can determine a set of medical tests and optionally parameters that may be required in order to diagnose the future medical condition in the specific person. Embodiments of the system may also determine sets of medical tests and optionally other parameters (not included in the medical tests) that may suit groups of people. For example, the system may determine such a set for predicting gestational diabetes mullites during pregnancy in Catholic, Hispanic women, in a specific age range (e.g., between 25 and 35 years of age).

Some embodiments may include methods for using EMR data and overcoming the sparse nature of such data. In some embodiments, a process of completing the sparse data by adding at least a portion of missing data may be performed using cross-validation analysis. The cross-validation process may be conducted in order to determine which data element or parameter is reliable and can be used to complete the data and which is not so. In some embodiments, the completed data may be analyzed in order to select the required parameters. The required parameters may than be utilized to train a ML model to predict a medical condition and to use the ML model to predict a future medical condition (e.g., hereinafter a precondition) in a patient who may not yet have any known or common symptom that may be indicative of or related (directly or indirectly) to the medical condition. A system and method according to some embodiments of the invention may be trained to predict a pregnancy related condition, for example, a gestational diabetes mullites (GDM), preeclampsia, preterm, hypothyroidism, hyperthyroidism, cardiovascular diseases, postpartum hemorrhage, fetal heart disorder, fetal growth disorder and intra-uterine growth restriction (IUGR) in a woman who is not pregnant at the time of prediction. In yet another example, embodiments of the system may predict additional medical conditions or preconditions such as hypothyroidism, bipolar disorder.

In the medical world methods of predicting a future medical condition are usually based on known medical connections between the condition and the tests. These known connections have been set by human researchers. However, researchers are normally subjected to prejudices and conceptions, and may thus look for connections between the condition and the tests in areas previously investigated and parameters that are known to be connected to medical conditions. A system according to embodiments of the invention may be free from such prejudices and conceptions, and may thus find connection that may be considered by human researchers as “nonlogical”, for example, the influence of a woman's religion or occupation on her chance to develop GDM during pregnancy.

A system according to embodiments of the invention may perform a bidirectional process starting from the final diagnosis of a medical condition going backwards, step by step finding the required parameters for predicting the medical condition. The system may then go forward starting from the required parameters to find the probability of having the medical condition. The system may use the predicting the medical condition in further training, as disclosed herein below.

As used herein, the term ‘EMR’ may relate to all systematized collection of patient and population health information in a digital format. Thus, EMR recorded data can be automatically received from databases associated with medical care providers, clinical institutes, medical insurance companies and the like. In some embodiments, EMR data may be manually uploaded into a system (such as system 100 disclosed herein below) via a user interface. In some embodiments, EMR data may be received from wearable devices, Telemedicine, IoT devices and the like. In some embodiments, EMR data may include managerial and financial data such as billing codes (i.e. ICD-9, ICD-10), medical staff, and the like.

As used herein, a “patient” may be a human, a pet and the like. In some embodiments, the patient may be associated with personal EMR data, that may be gathered and stored during the patient's visits to doctors, hospitals and the like.

As used herein, a “medical condition” may be an abnormal condition of a patient that may include, illnesses, diseases, lesions, disorders and the like. According to some embodiments, the medical condition may be diagnosed using any known medical diagnosis methods, such as, diagnostic imaging (e.g., ultrasound, X-ray, MRI, CT, etc.), medical laboratory test (e.g., blood tests, urine tests and the like) and diagnoses provided by doctors.

As used herein, an “appearance of the medical condition” may relate to a time during the life of the patient at which known or common symptoms related (directly or indirectly) to a specific medical condition may begin to appear. For example, an appearance of congestive heart failure (CHF) may include a time at which one of the symptoms of CHF (e.g., shortness of breath, excessive tiredness, leg swelling and the like) may initially appear.

As used herein, a “precondition” may be a future medical condition that may not yet be diagnosed or suspected and/or may not have yet showed or manifested any known or common symptoms that may be related (directly or indirectly) to the medical condition. For example, a precondition may be a pregnancy-related medical condition (e.g., GDM, preeclampsia, etc.) in a non-pregnant woman, development of a future hypothyroidism at adulthood in a teenager, a condition in which a person at his/her 30s will develop high blood pressure levels by the time they will reach their 50s, and the like.

As used herein a “decisive date” (DD) may be a date of importance or significance for the purpose of diagnosis or prediction of the medical condition. For example, the DD may be the date of recorded appearance of symptoms related (directly or indirectly) to the specific medical condition. In yet another example, for pregnancy the DD may be the last menstrual period (LMP).

As used herein, a “diagnosis of the medical condition” may relate to a definite establishment of knowledge that a patient is having the medical condition. In some embodiments, the diagnosis may be supported by “objective” tests, such as laboratory test (e.g., glucose tests for detecting diabetes), MRI scans for detecting tumors and the like.

As used herein, the term “sparse data” may relate to data or a data set that may lack values or information pertaining to one or more parameters. For example, with respect to EMR, sparse data may include parameters that are nullified (e.g., having zero values), missing values, missing varied parameters for different patients, thus having large diversity within the data. Sparse data, or the “curse of multidimensionality” problem is: the number of available data is comparable and often even superior to the number of patients. While the number of objects for statistical recognition procedures must be theoretically the square of the number of symptoms/medical conditions—in the reality, it the opposite: hundreds of symptoms and dozens of patients.

As used herein a “machine learning (ML) model” also known as Artificial Intelligence (AI) may refer to an information processing paradigm that may include: Supervised Learning, Unsupervised Learning, Semi-Supervised Learning, Reinforcement Learning, Deep Learning, Neural Network and the like.

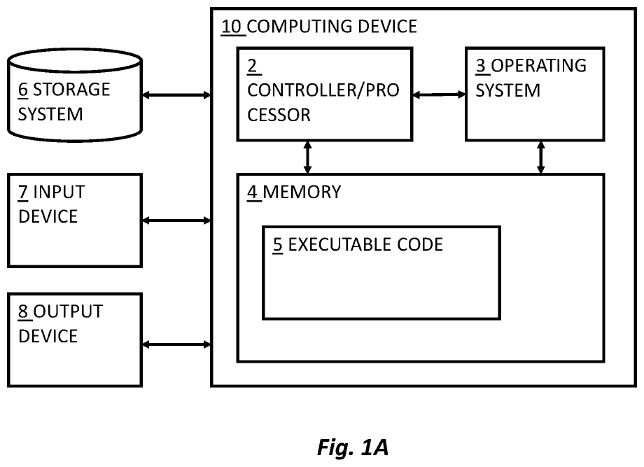

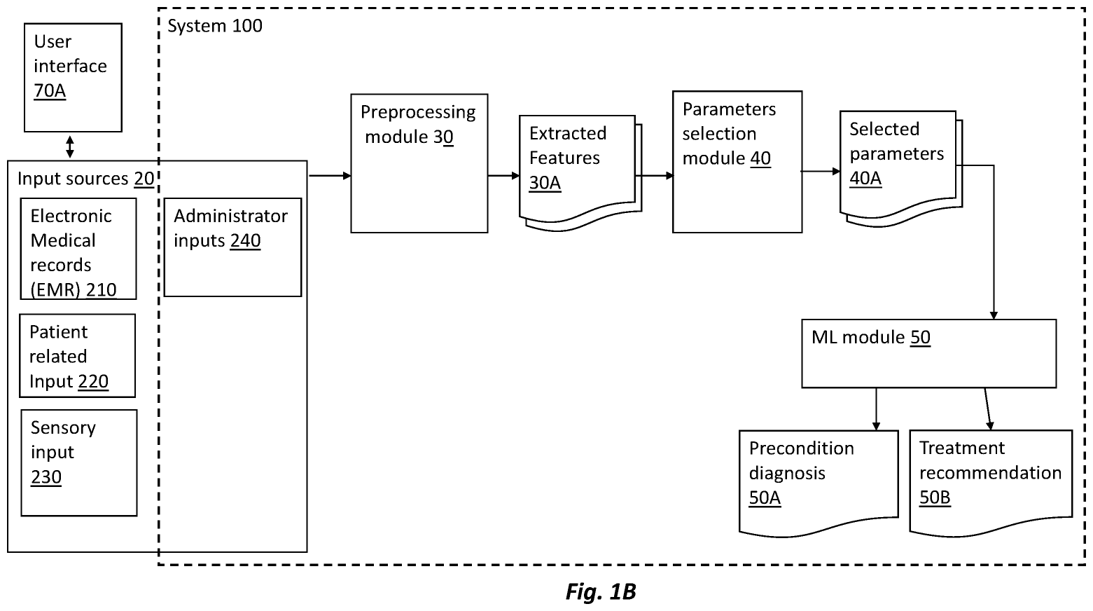

Reference is now made to FIG. 1A, depicting a computing device which may be included in a system according to some embodiments.

Computing device 10 may include a controller 2 that may be, for example, a central processing unit (CPU) processor or Graphical Processing Unit (GPU) processor, a chip or any suitable computing or computational device, an operating system 3, a memory 4, executable code 5, a storage system 6, input devices 7 and output devices 8. Controller 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of Computing device 1, for example, scheduling execution of software programs or tasks or enabling software programs or other modules or units to communicate. Operating system 3 may be a commercial operating system.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of, possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by controller 2 possibly under control of operating system 3. For example, executable code 5 may be an application that may perform execute machine learning or neural network applications for predicting a medical condition, etc. as further described herein. Although, for the sake of clarity, a single item of executable code 5 is shown in FIG. 1A, a system according to some embodiments of the invention may include a plurality of executable code segments similar to executable code 5 that may be loaded into memory 4 and cause controller 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Content may be stored in storage system 6 and may be loaded from storage system 6 into memory 4 where it may be processed by controller 2. In some embodiments, some of the components shown in FIG. 1A may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or Graphical Processing Unit (GPU) processor or any other suitable multi-purpose or specific processors or controllers (e.g., controllers similar to controller 2), a plurality of input units, a plurality of output units, a plurality of memory units, and a plurality of storage units.

Reference is now made to FIG. 1B, which is a block diagram, depicting a system 100 for performing any task and/or method according to embodiments of the invention, for example, at least one of: selecting required parameters for prediction or detection of a medical condition or precondition; predicting a pregnancy related medical condition in a non-pregnant woman; and determining a physician's profile. System 100 may assist physicians to accurately diagnose the patient and to provide a correct required treatment to the patient. System 100 may be or may include at least one computing device (e.g., element 10 of FIG. 1A), including one or more processors or controllers (e.g., element 2 of FIG. 1A), adapted to perform one or more embodiments of methods of the present invention. System 100 may include one or more input sources 20, a preprocessing module 30 and a parameters selection module 40. In some embodiments, system 100 may further include an ML module 50.

According to some embodiments, system 100 may receive one or more data elements from at least one (e.g., each) of input sources 20. In some embodiments, system 100 may receive data from one or more EMRs 210 (e.g., from one or more medical databases) and/or sensory input 230 (e.g., wearable devices). According to some embodiments, system 100 may receive patient related Input 220 which may include data that may be uploaded manually (e.g., via input element 7 of FIG. 1A). Such information may, for example, be stored on one or more computing systems (such as element 10 of FIG. 1A) of a hospital, a clinic a healthcare center, a medical insurance company, wearable devises, telemedicine, IoT devices and the like.

EMR data 210 may include, for example, previous data and/or analysis (e.g., made by a physician, a healthcare professional, and the like) of at least one of a physical, medical, intellectual, social and/or mental condition of the patient. For example, EMR data 210 may include data pertaining to historic growth metrics (e.g., evolution of the patient's weight and/or height over time). In another example, EMR data 210 may include a medical history of diagnosed physical, cognitive and/or mental diseases that the patient may have been diagnosed with. In yet another example, EMR data may include laboratory tests and diagnostic imaging. In some embodiments, EMR data 210 may include sparse data.

As elaborated herein, system 100 may include or may be in communication with at least one computing device (e.g., element 10 of FIG. 1) that may include one or more input devices 7 and/or output devices 8, as elaborated in relation to FIG. 1A. For example, system 100 may include a computing device (e.g., element 10 of FIG. 1A) such as smartphone, and the one or more input devices 7 and/or output devices 8 may include at least one user interface (UI) 70A, such as a touchscreen, adapted to present information to a user, and obtain information therefrom.

Additionally, or alternatively system 100 may include patient related input 220 that may present to a patient and/or a care provider (e.g., via UI 70A) one or more structured forms, adapted to obtain information pertaining to the patient's physical condition, general wellbeing, lifestyle and the like. In some embodiments, the patient and/or a care provider (or a guardian thereof, such as their parents) may fill the one or more structured forms, to produce at least one structured input data element 220. System 100 may store the at least one structured input data element 220 (e.g., on a database, such as storage element 6 of FIG. 1A) for further analysis.

Additionally, or alternatively, system 100 may include or may be in communication with one or more sensors for providing sensory data 230, including for example: wearable sensors (e.g., thermometers, blood saturation sensors, blood pressure sensors, etc.), external sensors (e.g., digital scale, digital height station, etc.), environment sensing devices, and the like.

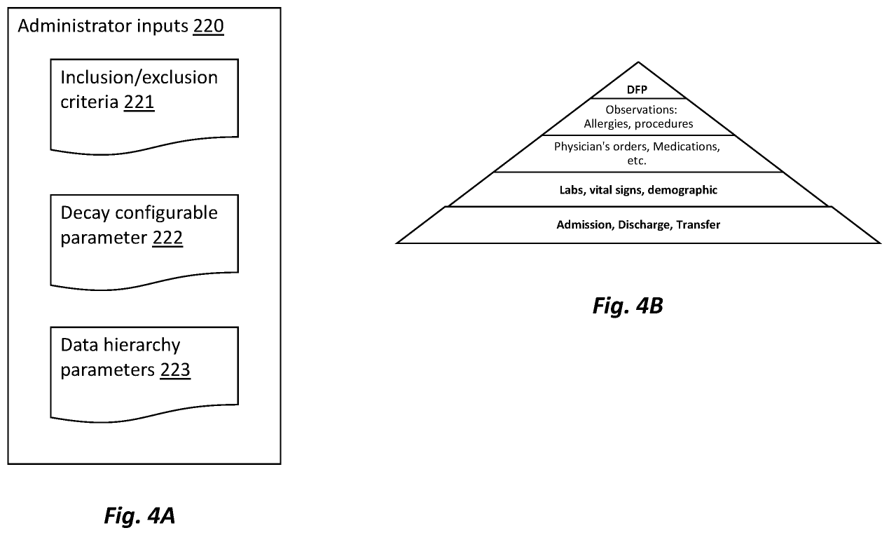

In some embodiments, system 100 may include administrator inputs 240, stored for example, in storage system 6. Administrator inputs 240 may include parameters provided to system 100 by an administrator. Examples for administrator input data elements 240 are elaborated herein (e.g., in relation to FIG. 4A). In some embodiments, administrator inputs 240 may include one or more inclusion/exclusion criteria 221 for including or excluding data that may be received from at least one of: EMR 210, user interface 70A, patient's input 220 and/or sensory input 230. For example, when training the system to predict GDM, the inclusion/exclusion criteria 221 may include: excluding data that may be received in relation to women that are diagnosed with type I or Type II diabetes, women that were never pregnant, patients that may have already been diagnosed with a condition that the system 100 may be trained to predict, and the like.

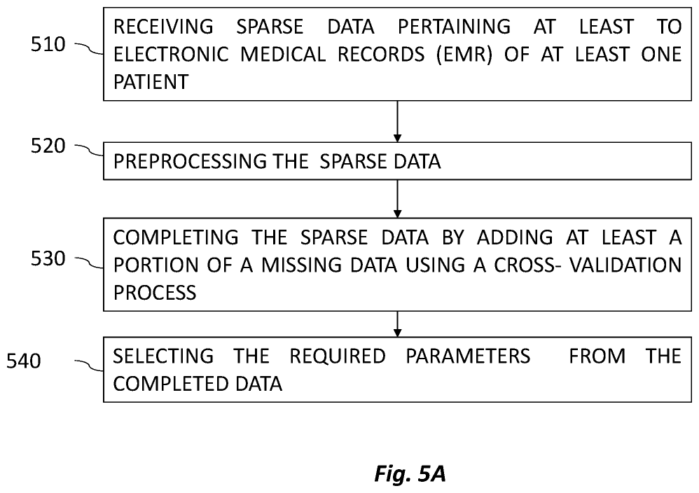

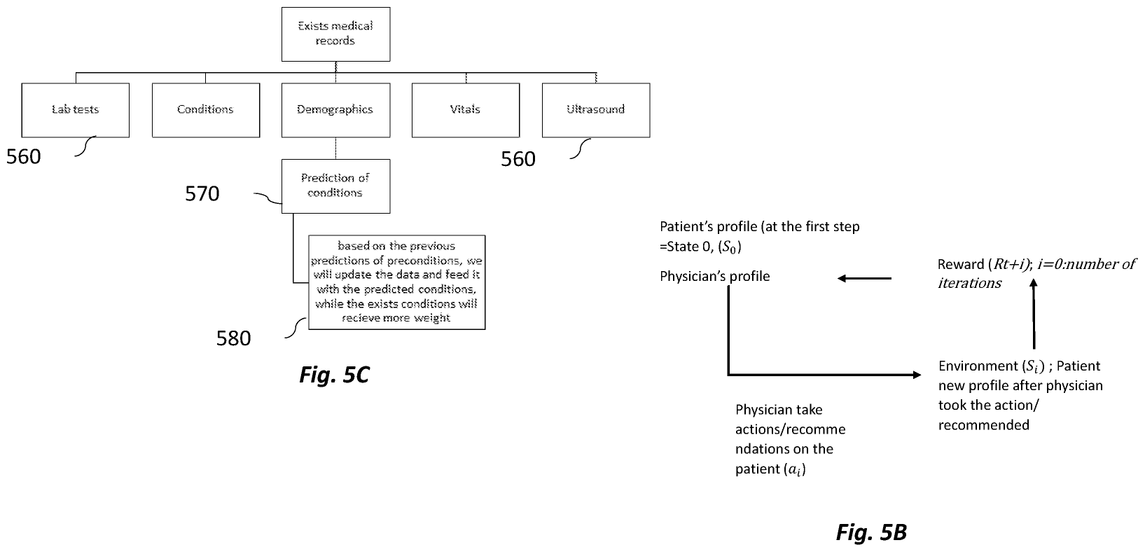

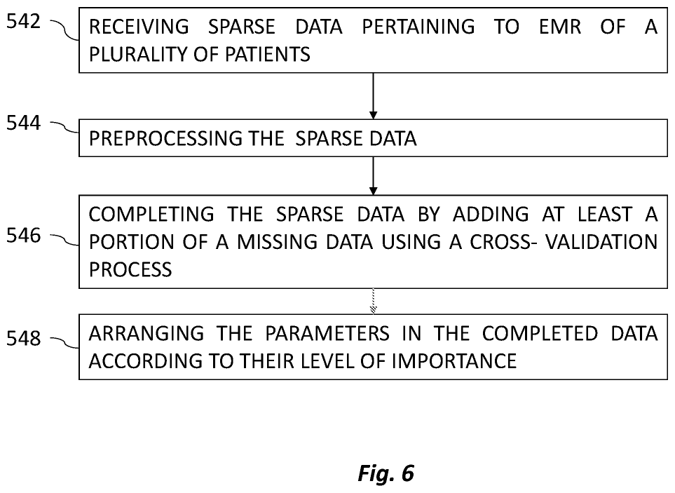

In some embodiments, administrator inputs 240 may include one or more configurable decay parameters 222 that may be used to apply weight to the various features that may be extracted from the received data. The configurable decay parameter 222 may be utilized to determine an influence of elapse of time (e.g., from a time at which a value of a feature or parameter has been receiving or obtained) on the prediction of the medical condition, as broadly discussed herein below (e.g., with respect to the methods of FIGS. 5 and 6).

In some embodiments, administrator inputs 240 may further include data hierarchy parameters 223, as illustrated in FIG. 4B. The lower the data in the illustrated pyramid the higher is the reliability and relevance of the data. For example, data received form detailed financial transactions (DFT) may be less relevant for prediction of a medical condition than observations and medication. According to some embodiments, the most reliable and relevant data may be data related to the admission, discharge, transfer (ADT) of patients from hospitals, and data form laboratory tests, vital signs and diagnostic tools such as diagnostic imaging.

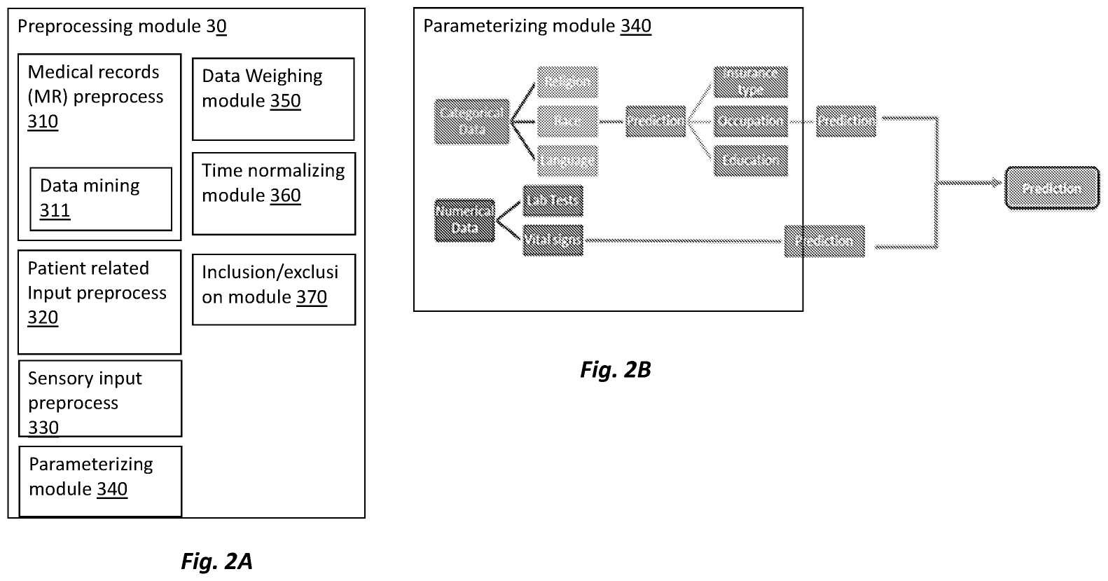

According to some embodiments, system 100 may include a preprocessing module 30. An elaborated depiction of preprocessing module 30 is provided in FIG. 2A. According to some embodiments, preprocessing module 30 may be configured to receive one or more data elements or parameter values from one or more input sources 20. The one or more data elements may include, for example: data pertaining to medical records from EMR 210; data pertaining to patient's input 220 (e.g., one or more profile data elements pertaining to the patient); data pertaining to sensory input 230 (e.g., real time measurements of height, weight, skin temperature and the like); and data pertaining to administrator input 240.

In some embodiments, preprocessing module 30 may include an EMR preprocessing module 310, configured to extract one or more features from EMR 210 data. For example, EMR preprocessing module 310 may be or may include a natural language processing (NLP) module 311, as known in the art. NLP module 311 may be configured to extract one or more features or data elements (e.g., words and/or phrases) that may be indicative of at least one of a historical, physical, intellectual, cognitive, social and/or mental condition of the patient, including for example: previous physical, cognitive, intellectual, behavioral, emotional, social and/or mental tests, examinations and/or treatments that the patient may have undergone, a previous diagnosis that has been provided by a physician or a healthcare professional and the like.

In some embodiments, preprocessing module 30 may include a patient's input preprocessing module 320. Patient's input preprocessing module 320 may be adapted to analyze input data 220 that may be or may include a form that may have been filled by the patient and/or by another person on their behalf (e.g., by a parent or a guardian). The form may include, for example, at least one data element pertaining to the patient profile and/or at least one data element pertaining to a suspected behavior and/or a developmental impediment which may be the subject of inquiry by system 100. In some embodiments, structured input preprocessing module 320 may include an NLP module as known in the art.

In some embodiments, preprocessing module 30 may include a sensory input preprocessing module 330, configured to extract one or more data elements or features from sensory input data 230. In some embodiments, sensory input module 230 may include or may be connected or associated with one or more sensors that may each be configured to sense a physical property (e.g., a movement) of the patient and/or their surrounding environment (e.g., an ambient temperature) and produce at least one data element pertaining to the sensed physical property.

In some embodiments, sensory input preprocessing module 330 may include inputs from real-time or updated laboratory tests conducted to the patients, that were not included in the patient's EMR. In some embodiments, sensory input preprocessing module 330 may include inputs from real-time or updated diagnostic imaging (e.g., ultrasound, X-ray, MRI, CT, etc.) that may not have been included in the patient's EMR.

In some embodiments, preprocessing module 30 may include parameterizing module 340 configured to provide a single value (e.g., a number, score, level, etc.) to each parameter in the EMR. An illustration of the parameterizing data received and processed by preprocessing module 30 is given in FIG. 2B.

In some embodiments, some parameters in the EMR may be defined by numerical data (e.g., a single value) and may herein be referred to as numeric parameters. Such parameters may include, for example, values of vital signs and laboratory tests indicating level of various compounds (e.g., in blood, urine etc.) of the patient.

Other parameters, may herein be referred to as categorial parameters, and may include, for example demographic parameters (e.g., religion, race, language, etc.) that may be difficult to define by a single value. Therefore, a scoring method for evaluating such categorial parameters may be included in parameterizing module 340. A nonlimiting example, for such scoring method is disclosed herein (e.g., with respect to FIGS. 10-15). Additional categorial parameters may include insurance type, occupation, education, marital status and the like. In some embodiments, parameterizing module 340 may be adapted to assign at least one (e.g., each) categorial parameters with a numerical value.

In some embodiments, parameterizing module 340 may be configured to processes monitored parameter values, that may be continuously monitored over time, including for example values pertaining to vital signs, blood pressure Holter and the like. In some embodiments, parameterizing module 340 may identify abnormalities in the monitored parameters and may select a value (e.g., maximum, minimum, slope etc.) that may represent this abnormality and the value of the monitored parameter.

Vital signs data that taken from hospitals and may include: HR, BP, SaO2, Temp., pain scale, respiratory rate, height, weight, BMI, etc. This data may be valuable since it may be used to predict complications, deterioration, and medical/physical conditions. The data may be collected from the hospital may be a labeled data, since it contains the physicians' diagnoses. In some embodiments, the monitored data may be taken only during the hospitalization, and therefore it may be very important to keep tracking the patients in home by connecting them various wearable devices. The drawback of using wearable devices is that it's very hard to make the labeling phase without using a team of doctors to recognize patterns in the data.

In some embodiments, parameterizing module 340 may use a novel and unique approach by using the labeled data from the hospital and transfer this learning to automatically label the wearable data. In some embodiments, system 100 may include a library of signals received from hospitals and labeled accordingly, and then label the data that was collected from wearable devices. For example, HR signals revived from hospitals, may be analyzed to detect an event of a heart attack. In addition, HR data collected from wearable devices for home use, may be analyzed to detect the same patterns as the ones recognized in the hospital as indicative of patient's heart attack.

In some embodiments, preprocessing module 30 may include a data weighing module 350. Data weighing module 350 may receive one or more configurable decay parameters 222 from administrator inputs 220 and may use these parameters to determine a time dependent weight W(t) of time dependent parameters, for example using equation 1. In some embodiment, each of the calculated W(t) may be used to weight at least one parameter, for example, the W(t) may multiply the parameter.

$$W ( t ) = {e}^{q ( t )} \qquad ( 1 )$$

Wherein: ‘q’ is a configurable parameter that determines the decay rate of the exponent. In some embodiments, t is the normalized time, set by a time normalizing module 360. In some embodiments, different ‘q’ s may be determined for different types of time dependent parameters, for example, using simulations and statistical methods.

In some embodiments, preprocessing module 30 may include a time normalizing module 360. Time normalizing module 360 may be configured to set a normalized time for one or more (e.g., all) patients that may be included in the EMR that were diagnosed with a specific condition.

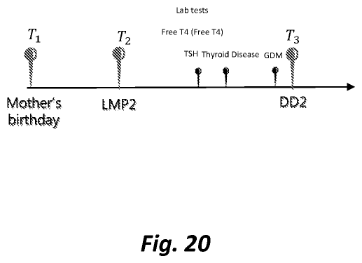

For example, a relative time may be taken from the patient's birth date. While the measurement of ‘t’ parameter may be done based on the difference between the previous sampling points in the data for the forecast moment. A nonlimiting example, for determining last menstrual period (LMP) for a woman is elaborated herein (e.g., with respect to FIG. 16 and the flowchart of FIG. 20). In some embodiments, a time for each time depended parameter (e.g., laboratory tests taken at a particular time) may be normalized with respect to the woman's LMP. The normalized time may be introduced into equation 1 for calculating the weight of each time-dependent parameter.

For example, time normalizing module 360 may utilize equation 2 for normalizing the data:

$$t = {( Converted )}_{Date ( dd , hh , mi )} = {DataPoint}_{Date} - D D \qquad ( 2 )$$

Where: the DataPoint_{Date} is the date at which the parameter was taken (e.g., date of laboratory test) or recorded (e.g., a diagnosis given by a doctor) and DD is the decisive date, for example, in GDM the DD is the moment that the woman came to consulting meeting, before or during the pregnancy.

In some embodiments, preprocessing module 30 may include an inclusion/exclusion module 370. In some embodiments, inclusion/exclusion module 370 may be used in a training phase of the method, as disclosed herein (e.g., with respect to step 544, of FIG. 6). Inclusion/exclusion module 370 may be configured to conduct an initial selection process of the EMR based on an inclusion/exclusion criteria. For example, for GDM prediction the exclusion criteria may include: women who diagnosed with one of the pre-pregnancy diabetes types; women who did not have any pregnancy in records; and women who were diagnosed after all their pregnancies.

In some embodiments, each patient included in the data may be tagged with a unique random index. In some embodiments, preprocessing module 30 may further be configured to: remove nulls; connect tables based on ‘p’ index, time, data types; remove duplications; connect data elements of each patient together from various sources and different sites, clinics and hospital; and dividing data types were to “islands” and, the system may performed a specified and unique analysis processes.

For example, when predicting GDM preprocessing module 30 may further be configured to: include only the data points before LMP, and during the pregnancy to refer only to the glucose values; taking, for woman having more than one pregnancy, data point also from time between the LMP and the date of diagnosis (DOD); and adding new column in tables with the pregnancy number.

In some embodiments, data features 30A may be received from preprocessing module 30 at parameters selection module 40 following the data preprocessing.

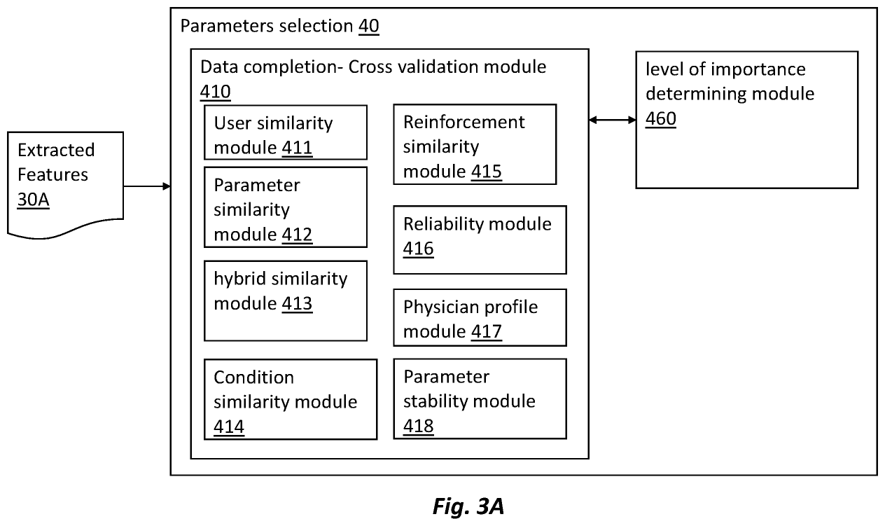

Referring now to FIG. 3, which is an illustration of A block diagram of parameters selection module 40 according to some embodiments of the invention. In some embodiments, parameters selection module 40 may include any module that may be configured to select required parameters out of the preprocessed EMR data provided by preprocessing module 30. Parameters selection module 40 may be configured to at least partially correct the sparsity of the preprocessed EMR data by completing at least some of data using a cross-validation process.

In some embodiments, the cross-validation process may include a plurality of similarity and reliability tests for ensuring the validity of the parameters used for completing the missing parameters. As should be understood by one skilled in the art, data completion-cross validation module 410 may include or may use at least some of modules 411-418. In some embodiments, only some of the modules may be used by system 100 at any required order. The invention is not limited to the disclosed order of the numerical references.

In some embodiments, cross validation module 410 of parameters selection module 40 may include a user similarity module 411 adapted to find similarity between patients' profiles to complete the missing elements in each profile. In some embodiments, the similarity may be calculated using different mathematical functions, for example, cosine centered, Euclidian distance, Manhattan distance and the like. In some embodiments, determining the similarity may be performed by mathematically calculating a distance or difference between different profiles and different parameters.

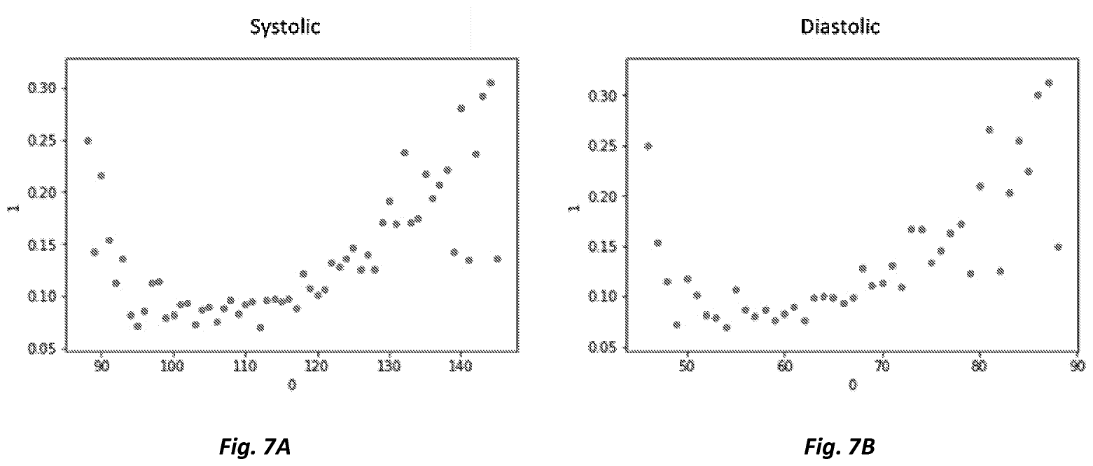

In some embodiments, cross validation module 410 may include a parameter similarity module 412 adapted to find similarity between parameters to complete the missing elements. In some embodiments, parameters selection module 40 may examine how a medical test (parameter) like heart rate is similar to blood pressure and/or in general similarity, as illustrated and discussed with respect to FIGS. 7A and 7b. In some embodiments, a similarity may be found between two different types of parameters, for example, a categorical parameter (e.g., Jewish religion) and a laboratory test (e.g., WBC). In some embodiments, parameter similarity module 412 may find that abnormalities in WBC have a higher probability to be found in people of the Jewish religion. In some embodiments, the similarity may be calculated using different mathematical functions, for example, cosine centered, Euclidian distance, Manhattan distance and the like. In some embodiments, determining the similarity may be performed by mathematically calculating the distance between different profiles and different parameters.

In some embodiments, cross validation module 410 may include hybrid similarity module 413 which finds the similarity of the relation between patients and medical tests (parameters) to see how it will influence on a specific condition/diagnosis. In some embodiments, determining the similarity may be performed by mathematically calculating a distance or difference between different profiles and different parameters, using any known method. The similarity processes are discussed in detail herein below with (e.g., in relation to the method of FIG. 5). In some embodiments, hybrid similarity module 413 may give a similarity score to each parameter based on the degree of similarity.

In some embodiments, cross validation module 410 may include condition a similarity module 414, adapted to find similarities between conditions (e.g., different diagnoses). In some embodiments, condition similarity module 414 may identify one or more (e.g., all) recorded conditions (e.g., all the diagnoses) included in the EMR and may for example, divide them to sub-groups based on at least one similarity (e.g., based on the type). For example, condition similarity module 414 may divide illnesses in women to sub-groups such as: pregnancy-related illnesses, fetus-related illnesses, woman health related conditions, general health conditions, etc.).

Condition similarity module 414 may use the conditions as the objective function (the objectives may be to predict/detect) and the medical tests (parameters) were used to describe the patient profile. This method of using different profiles with various conditions and objective functions may enable to understand the interactions between different parameters and connections. The prediction may be made on which patient's profile will develop one/more of the conditions.

In some embodiments, cross validation module 410 may include a reinforcement similarity module 415. Reinforcement similarity module 415 may be adapted to receive the determined similarities from at least one of modules 411-414 and feed the determined similarity back into at least one of the other modules, to optimize the process (e.g., to see if a better similarity is achieved). In some embodiments, reinforcement similarity module 415 may repeat at least some of the similarity processes in modules 411-414 to examine what is the most accurate and reliable result that can be received. The outcome of such optimization may be an enhanced performance of system 100.

In some embodiments, any one of modules 411-415 may use any known mathematical method for determining similarities. For example, any one of similarity modules 411-415 may further use matrix decomposition to reduce the dimension and divide the patients and/or the parameters into two separated matrices in order to find similarity. In some embodiments, different methods of matrix decomposition as LU, for instance, may be used when solving a system of linear equations Ax=b. The matrix A may be decomposed via the LU decomposition. The LU decomposition may factorize a matrix into a lower triangular matrix L and an upper triangular matrix U. The systems L(Ux)=b and Ux=L\hat{ }{{−1}}b require fewer additions and multiplications to solve, compared with the original system Ax=b, though one might require significantly more digits in inexact arithmetic such as floating point. In some embodiments, in order to examine which of the matrix decomposition method is the most accurate for each of the predictions, the system performed two steps”
Creating random “holes” (nulls) in the data, which may allow to compare between the predicted value (the value that the system completed while using one of the methods).
Comparing the matrix to a different matrix decomposition (as rank factorization, QR, etc.) to find what is the method that completes the data in the most accurate way, while the system compares it to the real elements.

In some embodiments, parameters selection module 40 may include a reliability module 416 for assessing the reliability of the data used for completing the missing elements. The term “reliability” may be used in this context to indicate a degree at which a first data elements may be utilized in order ensure that a parameter received from different ERM (e.g., test, or measuring procedure) yields the same results. In some embodiments, the reliability of each parameter may be determined based on the weight assigned for each parameter (e.g., in equation (1)) and the number of mutual parameters and may be calculated using equations (3) (e.g., first reliability level) and (4) (e.g., second reliability level)

$$R e l i a b i l i t {y}_{i} {\sum }_{n = 0}^{n = N - 1} W \qquad ( 3 )$$

Wherein N=Number of parameters, thus, the closer the analysis to the DD the higher the Reliability of the result.

$$R e l i a b i l i t {y}_{i i} = Number of mutual parameters \qquad ( 4 )$$

The total reliability (e.g., a reliability score) may be the function of the similarity and combination of Reliabilityᵢ and Reliabilityᵢᵢ.

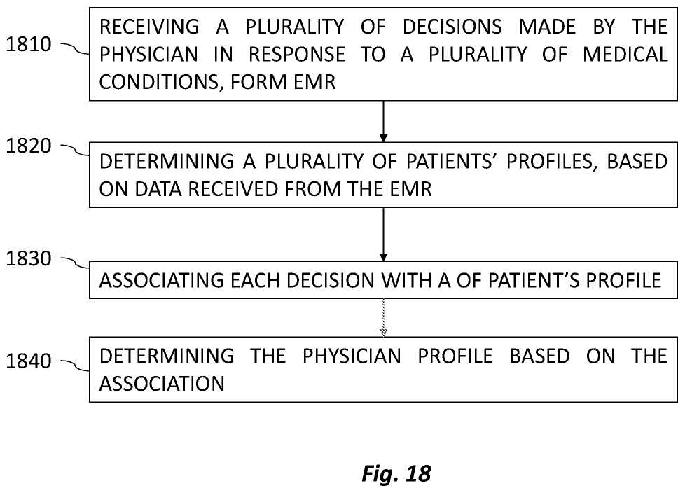

In some embodiments, parameters selection module 40 may include a physician profile module 417, which may be configured to estimate the reliability of the data received form a specific physician. A method of determining a physician profile is discussed herein (e.g., in relation to the flowchart of FIG. 18). In some embodiments, physician profile module 417 may be configured to minimize diversity in medical decisions (e.g., diagnoses, treatments and the like) made by physicians in identical situations. Human doctors are influenced by a variety of subjective elements that may affect their decisions. Such subjective element may include, inputs form different insurance companies, the personal chemistry between the patient and the doctor, the doctor's general mood at the decision time and the like. Physician profile module 417 may determine a decision diversity function, to correct the physician decision in identical medical situation. Physician profile module 417 may correct parameters related to physician inputs based on the determined decision diversity function. In some embodiments, physician profile module 417 may use mathematical and statistical methods to create the decision diversity function. The module may, for example, use any appropriate type of a neural network (NN) based ML model to determine or obtain the decision diversity function.

In some embodiments, cross validation module 410 may include a parameter stability module 418. Parameter stability module may distinguish between stable and non-stable parameters over time. For example, parameters such as religion, race, eye color may be related to as constant and stable, thus may be used to complete all the data related to a specific patient, for example, these parameters will be stable at any of her pregnancies. Nonstable parameters may include a financial state, a marital status and the like that may change between pregnancies, and may thus not be automatically used for completing missing data and may require additional inputs.

In some embodiments, parameters selection module 40 may be configured to complete the sparse data by adding at least a portion of a missing data using data that has received a sufficient similarity score and/or a sufficient reliability score. The similarity score and/or a sufficient reliability score may be calculated or determined using any combination of: user similarity module 411, parameter similarity module 412, hybrid similarity module 413, condition similarity module 414, reinforcement similarity module 415, reliability module 416, physician profile module 417 and parameter stability module 418.

For example, in treating GDM or general diabetes (given as nonlimiting example only) one goal may be to decrease the glucose levels. This may be defined as the decrease in percentages (trend) of the glucose levels. Another goal may be to balance the diabetes thus, may be label ‘resolved’ as a success, etc. In such case, the patients' profile may be divided into two groups—success or fail (it can be temporary success as well, like in glucose rate). Based on that, a patient profile may be assigned a success/fail label. In addition to the processes disclosed with respect to modules 411,-418 system 100 may further recommend a treatment based for example on physician profiles, the patient behavioral and epigenetic analysis—and optionally also the patient insurance type, socio-economic status, diet, smoking, use of drugs/alcohol and the like. For example, people in low socio-economic status probably have a poor diet, high stress, etc. All these may impact on the recommendation as well as the outcome. In some embodiments, physician profile module 417 may identify physicians that have a success and the physicians that failed.

For example, physician profile module 417 may identify a common trigger to provide the same treatment by the same physician to a specific patient's profile. For example, if the patient has high BMI, immediately this physician may give Insulin for suspected diabetes. Therefore, physician profile module 417 may create this connection between the patient's profile to the physician's profile. This connection may enable to recommend the physician on its own decisions that he is about to take, just by using the patient's profile. It has the potential to reduce the time during visits.

In some embodiments, parameters selection module 40 may the completed data to select the required parameters based on the entropy of the data. Parameters selection module 40 may include a level of importance determining module 460 for selecting the required parameters. In some embodiments, level of importance determining module 460 may be configured to determine the level of importance of the parameters using any combination of any feature importance methods known in the art, including for example, a decision tree, an NN-based ML model, Boots methods and the like.

In another example, level of importance determining module 460 may be configured to conduct information gain process to determine the level of importance based on how much information can be received from each data point (e.g., parameter). The information gain process may include the following equations:

$$EntropyE ( \vec{d} ) = - {\sum }_{i = 1}^{k} {p}_{i} {log}_{2} ( {p}_{i} ) \qquad ( 5 )$$

Wherein, EntropyE(\vec{d}) is the entropy of the patients, vector ‘d’ represent the total number of patients divided into sub-groups and each sub-group

$$\begin{matrix}{p}_{i} = \frac{number of patients in the sub \text{-} gruop}{total number of patients} . & \\ Entropy for a parameterE ( \vec{d} , \vec{a} ) = {\sum }_{j = 1}^{m} ( \frac{{n}_{j}}{n} \times E ( \vec{{d}_{J}} ) ) & ( 6 )\end{matrix}$$

Wherein ‘a’ is a vector of a specific parameter (e.g., a specific laboratory test) ‘n’ the number of patients associated with the parameter (e.g., the number of patients having a specific laboratory test) and ‘nⱼ’ the number of patients associated with the parameter that were diagnosed with the condition.

The information gain may be calculated by:

$$Information Gain I ( \vec{d} , \vec{a} ) = E ( \vec{d} ) - E ( \vec{d} , \vec{a} ) \qquad ( 7 )$$

The calculated information gain can be normalized to avoid bias using:

$$Relative Information Gain = \frac{Information Gain}{- {\sum }_{j = 1}^{v} {p}_{j}^{′} {log}_{2} ( {p}_{j}^{′} )} \qquad ( 8 )$$

A nonlimiting numerical example for calculating the information gain of bile acids laboratory test for woman diagnosed with GDM is given herein below.

All nonpregnant woman having laboratory tests were include in the main group and were divided into two sub groups: a first group of women having GDM, and a second group of women that do not have GDM:
Total number of 477987 women were included in the main group.

Total number of 64399 of women were diagnosed with GDM, therefore,

$$\frac{64399}{477987} = 0 .1343$$

Total number of 413588 of women were not diagnosed with GDM, therefore,

$$\frac{413588}{477987} = 0.86527$$

Accordingly,

$$E ( \vec{d} ) = - ( \frac{64399}{477987} {log}_{2} ( \frac{64399}{477987} ) + \frac{413588}{477987} {log}_{2} ( \frac{413588}{477987} ) ≅ 0. 5702$$

Calculation of entropy for Bile acids tests
From the total number of 477987 women, 49 women had tests of Bile acids
42 patients that had Bile acids test were healthy

$$\frac{42}{49} = 0.857$$

7 patients that had Bile acids test were diagnosed with

$$GDM \frac{7}{49} = 0.142$$

Accordingly,

$$E ( \vec{d} , Bile acids ) = ( \frac{49}{177987} \cdot 0.401 + \frac{477938}{477987} \cdot 0.19 ) = 0.1906$$

The information Gain→I(\vec{d}, Bile acids)=0.5702−0.1906=0.3796

In some embodiments, for each parameter the relative information gain may be calculated. In some embodiments, the calculated information gain may be used to determine the level of importance of each parameter. For example, the higher the information gain the higher is the level of importance/relevance.

Referring back to FIG. 1B, in some embodiments, system 100 may further include an ML module 50 that may be or may include any machine learning model known in the art. In some embodiments, the required parameters and/or the completed data may be provided to ML module 50, so as to train ML module 50 to predict a medical condition or precondition.

In some embodiments, trained ML module 50 may be configured to predict 50A a medical condition (precondition) before the appearance of the condition and/or to provide a treatment recommendation 50B.

Reference is now made to FIG. 5A which is a flowchart of a method of selecting, by at least one processor, required parameters for prediction or detection of a medical condition or precondition. The method of FIG. 5A may be executed by at least one processor or controller 2 (e.g., controller element 2 of FIG. 1A).

In step 510, the processor or controller 2 may receive sparse data originating from at least one EMR, and pertaining to at least one patient. The sparse medical data may be received from EMR 210. In some embodiments, additional data may be added to the sparse medical data, for example, from patients input 220 and sensory data 230.

In step 520, the at least one processor 2 may preprocess the sparse data, for example, using preprocessing module 30. In some embodiments, the preprocessing may include using EMR preprocess 310 including data mining 311, in order to extract data from the EMR. Additionally, or alternatively, the preprocessing may include using patient's input 220, using patient's input processes 320 and/or sensory data 230, using sensory input preprocess 330.

According to some embodiments, the data mining may include extracting numerical parameters such as laboratory tests, genetic tests, monitored charts and the like. Additionally, or alternatively, the data mining may include identifying similar categorial variables, such as free text from diagnoses, religion, race, ethnicity and the like. In some embodiments, a “Hard” clustering may be performed on every laboratory test and demographic variable for each demographic parameter, in order to unit between the options and to receive the real number of cases per option and per parameter. For example, varied appearances of parameter names like capital/small letters, spaces, etc., parameters that contained spelling errors, data that includes initials (e.g. MediCare, MCD, HGB, Hemoglobin, etc.). In some embodiments, the data mining may further include converting units to unified unit type. The conversion may be performed based on STD and mean, calculations and graphs, that may be compared with different codes and results to accurately assigned them to the appropriate ranges.

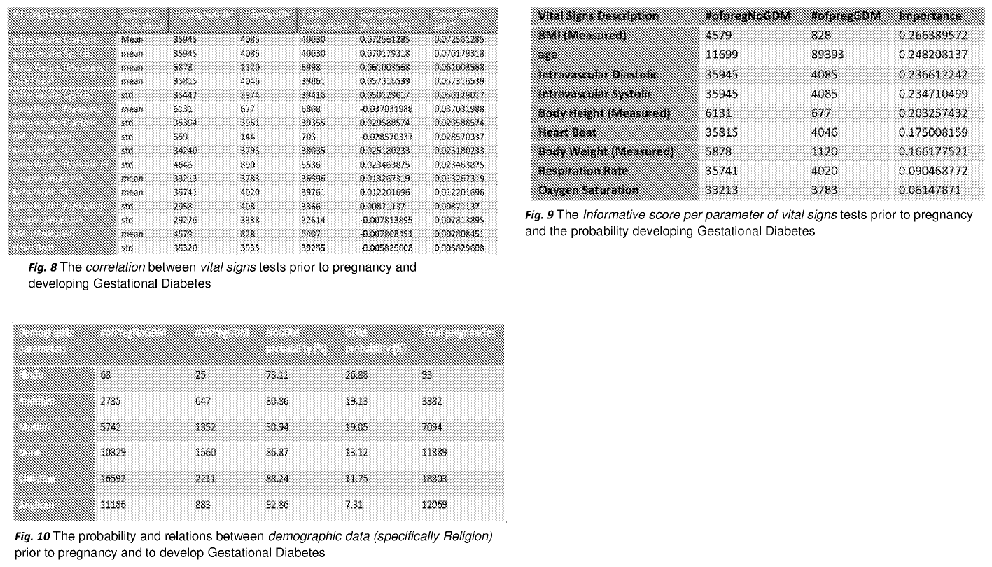

In some embodiments, the preprocessing may include assigning value to each parameter, using parameterizing module 340. For example, the processor may detect an abnormal behavior (e.g., a maximum or change) in a monitored signal, such as, vital signs and use the detected abnormal behavior to assign a representing value (e.g., a numerical value) to the detected at least one abnormal numerical value for the monitored parameter. In some embodiments, a score may be given to various categorical parameters, such as, demographic data, as discussed with respect to FIGS. 8-15. In some embodiments, the at least one category may include: a religion, an ethnicity, a race, a spoken language, an age, a gender, a citizenship, a social economic level, an insurance type, a level of education, a type of occupation, a place of birth and a place of living.

In some embodiments, the at least one categorical parameter may include one or more of a genetic parameter and/or an epigenetic parameter. In some embodiments, the genetic parameters may include the genetic profile of the patient, for example, by analyzing DNA results (e.g., WES, RNA, mRNA, etc.). In some embodiments, epigenetic parameters may include, lifestyle as diet, physical activity, behavioral aspects that may also influence the potential success of a specific treatment based on specific patient profile.

In some embodiments, the lifestyle's patient (e.g., diet, physical activity, smoking, alcohol, occupation, education, socio-economy status etc.) may reflect in its epigenetics. By using DNA results/by analyzing DNA results (WES, RNA, mRNA, etc.), and connecting them with the lifestyle data and lifestyle monitoring the patient's “vector” may be predicted—As it's in the mathematical and physics use the vector described by size and orientation—in the human body the genetics combined with epigenetics may be pointing on the patient's medical status, meaning the patient risks, conditions and preconditions. For example, if a patient has a gene of obesity, and he is keeping on healthy personalized lifestyle: fit diet, physical activity, etc., The system can determine differently its risk to obesity. If this patient with a familial background, e.g., genetics background of obesity, will have a non-healthy lifestyle it may enlarge the vector that proceeds to obesity and increase his risk.

In some embodiments, using various of monitors and records of lifestyle, like wearable devices, etc. for preforming a behavioral prediction. That may be used for several cases such as: prediction of recommendation/treatment success—if medical staff/trainer—is recommending on changing lifestyle, one patient may keep on the training plan for one week, the other may keep on 1 year, and for the third the physical activity can't be a “wrong” recommendation, since he/she will not do anything. In order to predict the expected behavior, the system may connect between the given orders and the patient/man/woman, and by doing that the system predicts the expected success of a recommendation on this specific person. On another use, the system used this to recommend on a treatment/order/recommendation also based on its personalized behavioral algorithm. Another option may be given by combining between the DNA analysis, the epigenetic prediction and the behavioral algorithm.

In some embodiments, the preprocessing of the sparse data, for training system 100, may include inclusion of data received only from patients diagnosed with the medical condition, based on one or more inclusion criteria. Examples for training system 100 are elaborate herein (e.g., in relation to the method of FIG. 6). In some embodiments, the one or more inclusion criteria may include inclusion of the data received from patient in precondition, for example, before the medical condition was diagnosed, suspected and/or showed any known/common symptoms related to (directly or indirectly). The sparse data may be filtered by, for example, inclusion/exclusion module 370 using inclusion/exclusion criteria 221.

A nonlimiting example for the use, during the training stage of the system, of inclusion inclusion/exclusion criteria on sparse data for women with GDM is given in table 1, which shows excluding data received from women having type I or Type II diabetes. Since the prediction purpose of the given example is to predict GDM, the women that were already diagnosed with diabetes needs to be excluded from this data set. The GDM was identify from glucose levels, for example, from free text Glucose levels >85 mg/dL, billing records (e.g., ICD-9/10 codes) and the like. In the following process, 826 women diagnosed with one of the pre-pregnancy diabetes types, one woman how did not have any pregnancy in records, 140 women were diagnosed after all their pregnancies, and kept and used all their pregnancies in the data, 685 women who diagnosed with one of the above, and all the pregnancies were removed that were after the diagnosis time.

TABLE 1

| | # of pregnancies | # of pregnancies |
|---|---|---|
| # of patients | Before removal | After removal |
| 410 | 1 | 0 |
| 81 | 2 | 0 |
| 107 | 2 | 1 |
| 20 | 3 | 0 |
| 13 | 3 | 1 |
| 23 | 3 | 2 |
| 6 | 4 | 0 |
| 1 | 4 | 1 |
| 5 | 4 | 2 |
| 9 | 4 | 3 |
| 1 | 5 | 1 |
| 2 | 5 | 3 |
| 1 | 5 | 4 |
| 1 | 6 | 3 |
| 3 | 6 | 4 |
| 1 | 6 | 5 |
| 1 | 7 | 4 |

In some embodiments, at least one categorical dependent parameter may be a geographic parameter related to a location of the patient selected from: radiation levels at the location, temperatures at the location, humidity levels at the location and altitude of the location. Each one of these parameters may affect the medical condition, thus may assign with a different score. In some embodiments, the geographic parameters may be used to define the mathematical relations and functioning between the medical condition and the geographic parameter.

In some embodiments, the processor may normalize time dependent parameters in the sparse data received from a patient's lives to a single timeline, using for example, time normalizing module 360. In some embodiments, the processor may use equation (2) to normalize the date of each time dependent parameter with respect to the DD. In some embodiments, the DD may define a timeline for each type of time-dependent parameter, for example, for a specific type of laboratory test the timeline may be the time from conducting the first laboratory test of to the DD. The timeline may include the dates and respective value of all the tests of the specific type conducted during the timeline.

In some cases, distinguishing between the potential influences of different periods in life on a specific condition, is crucial. For example, for some medical conditions, tests and parameters taken during adolescence may have higher impact than parameters taken during adulthood. In order to address this issue, parameters received from different periods of time may be assigned with different decay parameters (of equation (1)) for different periods.

In some embodiments, the timeline may be divided into two or more time intervals. For example, Body Mass Index (BMI) measurements of a woman may be divided to childhood ages 0-12, adolescence ages 12-20, ages 20-30 and adulthood (e.g., 30+). In some embodiments, each time dependent parameter may be associated with a specific time interval. For example, all BMI measurements may each be associated with the specific time interval at which the measurement were taken. In some embodiments, a specific weighing process may be conducted, using for example, data weighing module 350, for the parameters in each time interval.

For example, different configurable decay rate parameter ‘q’ may be determined to different time intervals. Therefore, the weight of each time dependent parameter may be calculated, by equation (1) using the corresponding decay rate parameter. Accordingly, for each time interval a specific equation (1) may be determined. For example, BMI measurements during the adolescence ages, may receive extra weighing while predicting a condition that affects fertility issues like polycystic ovaries syndrome (PCO).

In step 530, processor 2 may complete the sparse data by adding at least a portion of a missing data using a cross-validation process. For example, processor 2 may use any one of: user similarity module 411, parameter similarity module 412, hybrid similarity module 413, condition similarity module 414, reinforcement similarity module 415 reliability module 416, physician profile module 417, and parameter stability module 418 to evaluate the similarity of different parameters of different users and to determine the reliability of each parameter. Therefore, the cross-validation process may allow to select for completion of missing data only the one having sufficient reliability and similarity. In some embodiments, the system and method may reenter similar and/or reliable parameters, conditions and/or users found in one of the modules 411-418 back into one of the other modules in order to find further similarities and check the mutual influences between the found similarities and reliabilities. Such a back and forth method may allow to increase the performance of system 100 (e.g., the completion of missing data and further the detection/prediction of the condition).

In step 540, the processor may arrange and then select the parameters in the completed data according to their level of importance. In some embodiments, the processor may use level of importance determining module 460, illustrated in FIG. 1A, to determine the level of importance of each parameter. The higher the level the more important is the parameter for the prediction of the medical condition. The list of parameters may include, for example: parameters related to medical tests (e.g., laboratory tests, monitored signals, diagnostic imaging and the like) and personal parameters (e.g., weight, height, categorical parameters and the like). A method of electing the required parameters based on level of importance is discussed herein (e.g., with respect to FIG. 6).

Reference is now made to FIG. 6 which is a flowchart of a method of selecting the required parameters is by a module, for example, level of importance determining module 460. Steps 542-546 may be substantially the same as steps 510-530 of the method of FIG. 5A, conducted on a plurality of patients. In some embodiments, processor 2 may select only patients diagnosed with the medical condition and use their data, using for example, inclusion/exclusion module 370. In step 548, the processor may arrange the parameters in the completed data according to their level of importance, for example, using level of importance determining module 460.

In some embodiments, processor 2 may identify in the required parameters, one or more parameters related to medical tests and may determine a list of required medical tests for prediction or detection of a medical condition based on the identified parameters. For example, processor 2 may select to include in the list only parameters having level of importance that may be higher than a threshold value. In some embodiments, the processor may be configured to arrange the parameters and/or determine a list of required medical tests for a specific patient. In some embodiments, the processor may be configured to arrange the parameters and/or determine a list of required medical tests for a group of patients having at least one similar personal parameter, for example, a categorical parameter.

A nonlimiting example, of assessing and scoring the categorical parameter of religion with respect to the diagnosis of GDM is given in the tables below.

In some embodiments, the above may be used to evaluated whether there were any relations between a combination of parameters and the probability of developing GDM. In some embodiments, the number of previous pregnancies was combined with the history of GDM as well as the birth order (e.g. the difference of probability between a patient with three prior pregnancies with a history of GDM in her first pregnancy to another patient with three prior pregnancies with history of GDM in her last pregnancy). The results are shown in Table 2. It was found that the birth order of the history of gestational diabetes affects the probability of having GDM in the current pregnancy. For example, a patient entering her fourth pregnancy with two prior pregnancies complicated by gestational diabetes where her last pregnancy did not have gestational diabetes had a lower probability of developing GDM compared to another patient with the same number of pregnancies and history of pregnancies complicated by gestational diabetes, but the last pregnancy was complicated by gestational diabetes (20.7% versus 45.5%, respectively).

Table 2 shows the probability of developing GDM as function of religion taking into account previous pregnancies.

TABLE 2

| | Total | Number of | Number of | |
|---|---|---|---|---|
| | number of | pregnancies | pregnancies | GDM* |
| | pregnancies | with GDM* | without GDM* | probability |
| Religion | [N] | [N] | [N] | [%] |
| Hindu | 143 | 24 | 119 | 16.8 |
| Pentecostal | 175 | 25 | 150 | 14.3 |
| Seventh Day | 92 | 13 | 79 | 14.1 |
| Adventist | | | | |
| Muslim | 7,611 | 1,062 | 6,549 | 14.0 |
| Protestant | 61 | 7 | 54 | 11.5 |
| Buddhist | 901 | 103 | 798 | 11.4 |
| Baptist | 288 | 32 | 256 | 11.1 |
| Catholic | 6,703 | 703 | 6,000 | 10.5 |
| Christian | 4,455 | 381 | 4,074 | 8.6 |
| Jehovah | 110 | 9 | 101 | 8.2 |
| Witness | | | | |
| Russian | 100 | 8 | 92 | 8.0 |
| Orthodox | | | | |
| Anglican | 51 | 2 | 49 | 3.9 |
| Jewish | 22,705 | 738 | 21,967 | 3.3 |

In some embodiments, the quality of the algorithm may be estimated using specificity, sensitivity, accuracy and/or loss, an example is given herein below in table 3. In some embodiments, the specificity may be defined as true positives while the sensitivity may be defined as true negatives. The accuracy may be equivalent to success rate; meaning, how many times an algorithm predicts correctly if a disease will develop. The loss may be a number indicating how accurate the algorithm's prediction was on a single example. In some embodiments, if the algorithm's prediction is perfect, the loss is zero; otherwise, the loss is greater. The goal of training a model (e.g., module 50) is to find a set of weights and biases at a minimum loss (<0.5), on average, across all examples.

Table 3 shows more specificity the specificity, sensitivity, accuracy and loss on two religion domains.

TABLE 3

| | Total | | | | |
|---|---|---|---|---|---|
| | number of | | | | |
| | pregnancies | | | | |
| Religion | (N*) | Specificity | Sensitivity | Accuracy | Loss |
| Catholic | 6,703 (703) | 98.1% | 88.0% | 93.0% | 0.0693 |
| Jewish | 22,705 (738) | 99.3% | 96.5% | 97.9% | 0.0207 |

Table 4 shows for all women a combination of birth order with history of GDM.

TABLE 4

| | Number of | | | |
|---|---|---|---|---|
| | prior | | | Probability to |
| | pregnancies | Last | Total | develop GDM* |
| Number of | with | pregnancy | number of | in current |
| pregnancies | GDM* | with | pregnancies | pregnancy |
| [N] | [N] | GDM* | [N] | [%] |
| 3 | 0 | 0 | 7,940 | 6.1 |
| 3 | 1 | 0 | 556 | 17.8 |
| 3 | 1 | 1 | 583 | 25.4 |
| 3 | 2 | 1 | 178 | 42.7 |
| 4 | 0 | 0 | 3,802 | 5.1 |
| 4 | 1 | 0 | 359 | 18.1 |
| 4 | 1 | 1 | 226 | 22.6 |
| 4 | 2 | 0 | 29 | 20.7 |
| 4 | 2 | 1 | 101 | 45.5 |
| 4 | 3 | 1 | 35 | 57.1 |

Some examples for lists of required medical tests to predicting a precondition of GDM in various groups of women are listed herein below in table 5.

TABLE 5

| | | | For women in |
|---|---|---|---|
| Jews | Muslims | Christians | general |
| ‘ANTI DNA | ‘COLLAGEN’ | ‘ANTI THROMB 3’ | ‘CHLORIDE’ |
| ANTIBODY DOUBLE | | | |
| STRAN’ | | | |
| ‘BETA-HCG | ‘FETAL HEMOGLOBIN’ | ‘CHENODEOXYCHOLIC | ‘D-DIMER |
| QUANTITATIVE’ | | ACID’ | HIGH’ |
| ‘BASE DEFICIT’ | ‘BILIRUBIN_TOTAL’ | ‘COXSACKIE B 5’ | ‘BAND |
| | | | NEUTROPHIL’ |
| ‘CALCIUM’ | ‘BASOPHIL’ | ‘B2 GLYCOPROTEIN | ‘LYMPH |
| | | IGA’ | ABSOLUTE’ |
| ‘EST GFR IF BLACK’ | ‘AMYLASE’ | ‘% Ly (Lymphocytes | ‘3 Hour (OGTT - |
| | | Percentage)’ | Three Instance)’ |
| ‘% Neu (Neutrophils | ‘BILE ACIDS_TOTAL’ | ‘Candida krusei’ | ‘CREATININE |
| Percentage)’ | | | REFERENCE |
| | | | LAB’ |
| ‘B2 GLYCOPROTEIN | ‘CSF NEUTROPHILS’ | ‘BASO ABSOLUTE’ | ‘LUPUS PTT’ |
| IGA’ | | | |
| ‘FACTOR II’ | ‘CMV IgG INTERP’ | ‘AMMONIA’ | ‘LITHIUM’ |
| ‘% Ba (Basophils | ‘% Mo (Monocytes | ‘ACANTHROCYTES’ | ‘HSV 2 IgM |
| Percentage)’ | Percentage - Manual WBC | | (value) (HSV 2 |
| | Differential)’ | | IgM Float)’ |
| ‘ANION GAP’ | ‘CMV IGM AB’ | ‘CPK’ | ‘L/S RATIO’ |
| ‘ASO TEXT’ | ‘DEOXYCHOLIC ACID’ | ‘DHEA’ | ‘EST GFR |
| | | | OTHER RACES’ |
| ‘CSF GLUCOSE’ | ‘% Eo (Eosinophils | ‘BUN’ | ‘FACTOR VII’ |
| | Percentage)’ | | |
| ‘% Eo (Eosinophils | ‘ANTI THROMBIN III | ‘Candida albicans’ | G-6-PD |
| Percentage)’ | ACTIVITY’ | | SCREEN’ |
| ‘FACTOR VIII’ | ‘ABO’ | ‘CARDIAC | ‘B2 |
| | | TROPONIN-CTNI’ | GLYCOPROTEIN |
| | | | IGA’ |
| ‘CHOLESTEROL’ | ‘CORD PO2_VENOUS’ | ‘CSF BANDS’ | ‘ANTI DNA |
| | | | ANTIBODY |
| | | | DOUBLE |
| | | | STRAN’ |
| ‘BILE ACIDS_TOTAL’ | ‘FACTOR VIII’ | ‘CREATININE | ‘CSF |
| | | PERIOD’ | ALBUMIN’ |
| ‘CALCIUM IONIZED’ | ‘% Mo (Monocytes | ‘ANTITHROMBIN’ | ‘HEMOGLOBIN |
| | Percentage)’ | | A2 (QUANT)’ |
| ‘% Band (Band | ‘Candida krusei’ | ‘B-TYPE | ‘FLUID |
| Percentage - Manual | | NATRIURETIC | LYMPHOCYTES’ |
| WBC Differential)’ | | PEPTIDE’ | |
| ‘COXSACKIE B 5’ | ‘BICARBONATE’ | ‘ALPHA FETO | ‘LYMPH |
| | | PROTEIN TUMOR | PERCENT’ |
| | | MARKE’ | |
| ‘COMPLEMENT C4’ | ‘COXSACKIE B 5’ | ‘AMYLASE’ | ‘GLUCOSE_FLUID’ |
| ‘B TITER_NONSP AG:’ | ‘ANISOCYTOSIS’ | ‘CREATININE | ‘DHEA’ |
| | | REFERENCE LAB’ | |
| ‘DHEA-S’ | ‘CDFT’ | ‘3 Hour (OGTT - Three | ‘CALCIUM’ |
| | | Instance)’ | |
| ‘Candida albicans’ | ‘CORD HCO3_ARTERIAL’ | ‘CSF RBC’ | ‘ACETYLCHOLINE |
| | | | RECEPTOR AB’ |
| ‘BETA-2- | ‘AMMONIA’ | ‘BILIRUBIN_TOTAL’ | ‘BASE |
| MICROGLOBULIN’ | | | EXCESS’ |
| ‘D-DIMER HIGH’ | ‘EST VOL FETAL | ‘BASOPHIL’ | ‘HOMOCYSTINE |
| | BLOOD’ | | NUTRITIONAL |
| | | | SERUM’ |
| ‘CORD HCO3_VENOUS’ | ‘ASCA IgA Interp’ | ‘% Ba (Basophils | ‘EBV VCA IGG |
| | | Percentage)’ | AB’ |
| ‘AMMONIA’ | ‘3 Hour (OGTT - Three | ‘D-DIMER’ | ‘BETA-2- |
| | Instance)’ | | MICROGLOBULIN’ |
| ‘CSF WBC’ | ‘ANTI-NUCLEAR AB | ‘AUTOFILING | ‘ENTEROVIRUS |
| | TITER’ | CONDITION’ | RNA_QL_RT-PCR’ |
| ‘% Mo (Monocytes | ‘CHENODEOXYCHOLIC | ‘CALCIUM IONIZED’ | ‘CDFT’ |
| Percentage)’ | ACID’ | | |
| ‘3 Hour (OGTT - Three | ‘ACTIVATED PROTEIN | ‘BASE DEFICIT’ | ‘GLUCOSE’ |
| Instance)’ | C RESISTANCE’ | | |
| ‘ALK | ‘AFP (Alpha Fetoprotein)’ | ‘CHOLESTEROL_SERUM’ | ‘CHLORIDE’ |
| PHOSPHATASE’ | | | |
| ‘AUTOFILING | ‘CREATININE VOLUME’ | ‘2 Hour (OGTT - Two | |
| CONDITION’ | | Instance)’ | |
| ‘ALBUMIN’ | ‘D-DIMER’ | ‘ACTIVATED | |
| | | PROTEIN C | |
| | | RESISTANCE’ | |
| ‘ESR (Erythrocyte | ‘CSF EOSINOPHILS’ | ‘BETA-2- | |
| Sedimentation Rate)’ | | MICROGLOBULIN’ | |
| ‘ALPHA FETO | ‘ASO TEXT’ | ‘COXSACKIE B 6’ | |
| PROTEIN TUMOR | | | |
| MARKE’ | | | |
| ‘FETAL LUNG | ‘1 Hour (OGTT - One | ‘Down Syndrome Risk | |
| MATURITY’ | Instance)’ | by Age: 1: (Down | |
| | | Syndrome Risk Age)’ | |
| ‘EOSINOPHIL’ | ‘CSF WBC’ | ‘AFP (Alpha | |
| | | Fetoprotein)’ | |
| ‘FLUID | ‘FACTOR VII’ | ‘BURR CELLS’ | |
| MONOCYTES’ | | | |
| ‘ASSIGNED VALUE’ | ‘ALK PHOSPHATASE’ | ‘ASO TEXT’ | |
| ‘C. TRACHOMATIS | ‘% ALBUMIN’ | ‘CREATININE_24 HR | |
| INTERPRETATION’ | | URINE’ | |
| ‘ANISOCYTOSIS’ | ‘FIO2’ | ‘BETA-HCG | |
| | | QUANTITATIVE’ | |
| ‘CREATININE | ‘CSF GLUCOSE’ | ‘DHEA-S’ | |
| VOLUME’ | | | |
| ‘EPI’ | ‘Antithyroid Peroxidase Ab | ‘DEOXYCHOLIC | |
| | (Anti TPO)’ | ACID’ | |
| ‘CREATININE_24 HR | ‘ALPHA FETO PROTEIN | | |
| URINE’ | TUMOR MARKE’ | | |
| ‘FERRITIN’ | ‘EBNA AB (IGG)’ | | |
| ‘CHENODEOXYCHOLIC | ‘CREATININE_BLOOD’ | | |
| ACID’ | | | |
| ‘DHEA’ | ‘CORD PH_ARTERIAL’ | | |
| ‘AMP/SULBACTAM’ | ‘B-TYPE NATRIURETIC | | |
| | PEPTIDE’ | | |
| ‘BODY | ‘CSF TOTAL PROTEIN’ | | |
| TEMPERATURE’ | | | |
| ‘COMPLEMENT C3’ | ‘COMPLEMENT C3’ | | |
| ‘ANTITHROMBIN’ | ‘BAND NEUTROPHIL’ | | |
| ‘CDFT’ | ‘ANTITHROMBIN’ | | |
| ‘ABO’ | ‘FERRITIN’ | | |
| ‘FACTOR VII’ | ‘Down Syndrome Risk 1: | | |
| | (Down Syndrome Risk)’ | | |
| ‘BASOPHIL’ | ‘Enterococcus species’ | | |
| ‘BLOOD HCG | ‘BETA-2- | | |
| QUALITATIVE’ | MICROGLOBULIN’ | | |
| ‘BASO ABSOLUTE’ | ‘FLUID BANDS’ | | |
| ‘CREATININE_FLUID’ | ‘BILIRUBIN_DIRECT’ | | |
| ‘CO2’ | ‘EBV VCA IGG AB’ | | |
| ‘CORD PO2_ARTERIAL’ | ‘Candida albicans’ | | |
| ‘COLLECTION TIME’ | ‘CSF MONOCYTES’ | | |
| ‘EOSIN | ‘CREATININE_24 HR | | |
| METAMYELOCYTE’ | URINE’ | | |
| ‘ENTERO/RV’ | ‘BLOOD HCG | | |
| | QUALITATIVE’ | | |
| ‘AST’ | ‘CKMB’ | | |
| ‘ACTIVATED | ‘COMPLEMENT_TOTAL’ | | |
| PROTEIN C | | | |
| RESISTANCE’ | | | |
| ‘ACANTHROCYTES’ | ‘ERTAPENEM’ | | |
| ‘1 Hour (OGTT - One | ‘2 Hour (OGTT - Two | | |
| Instance)’ | Instance)’ | | |
| ‘EBV VCA IGM AB’ | ‘CREATININE | | |
| | REFERENCE LAB’ | | |
| ‘CMV IgG INTERP’ | ‘CHLORIDE’ | | |
| ‘A TITER_CRYPT AG:’ | | | |
| ‘Down Syndrome Risk | | | |
| by Age: 1: (Down | | | |
| Syndrome Risk Age)’ | | | |
| ‘BURR CELLS’ | | | |
| ‘FLUID BANDS’ | | | |
| ‘CARDIAC | | | |
| HOMOCYSTEINE’ | | | |
| ‘BASE EXCESS’ | | | |
| ‘ESBL’ | | | |
| ‘DIGOXIN’ | | | |
| ‘CREATININE_{—} | | | |
| RANDOM URINE’ | | | |
| ‘BASO PERCENT’ | | | |
| ‘CORTISOL A.M.’ | | | |
| ‘% ALBUMIN’ | | | |
| ‘ENTEROVIRUS | | | |
| RNA_QL_RT-PCR’ | | | |
| ‘CREATININE_BLOOD’ | | | |
| ‘CHOLESTEROL_SERUM’ | | | |
| ‘CARDIAC | | | |
| TROPONIN-CTNI’ | | | |
| ‘ANTI-NUCLEAR AB | | | |
| TITER’ | | | |
| ‘EST VOL FETAL | | | |
| BLOOD’ | | | |
| ‘B2 GLYCOPROTEIN | | | |
| IGM’ | | | |
| ‘CMV IGM AB’ | | | |
| ‘CORD PH_VENOUS’ | | | |
| ‘Diabetes Screen | | | |
| (GCT)’ | | | |

In some embodiments, the above sets of tests may be included in a method of predicting one or more pregnancy-related conditions in a non-pregnant woman. In some embodiments, the method may include conducting a set of medical tests and identifying abnormalities (e.g., higher or lower levels out of the normal range or other suspicious levels) in at least one of the tests in each set of medical tests. In some embodiments, system 100 and the methods of FIGS. 5-6 may provide more than one set of tests for predicting the same medical conditions. Herein below are some examples for such prediction methods. As shown below, at least some of the parameters listed are ones that are not known in the medical art to be related or indicative in any way to the disclosed conditions. Such a list of parameters may be the result of a system such as system 100 and the methods of FIGS. 5-6.

The below methods may be applicable for both predicting a pregnancy condition on a nonpregnant woman and detecting the pregnancy condition in a pregnant woman.

In some embodiments, a first method of predicting/detecting GDM in a woman may include: receiving the at least 5 of the following laboratory test results, obtained from the woman: ACETONE, HEMOGLOBIN S, FREE T4, URINE_GAMMA GLOBULIN, TSH THIRD GENERATION, METAMYELOCYTE, TEAR DROP CELL, PROGESTERONE, HEP B SURFACE AG, PHOSPHOROUS, ESBL, BETA-HCG QUANTITATIVE, URINE KETONES, TOXOPLASMA IGM, RBC, BAND NEUTROPHIL, METANEPHRINES TOTAL, LYMPHOCYTE, TOTAL T3, Na (Sodium), NRML_TEXT, FREE T3, MYELOCYTE, URINE Diabetes Screen (GCT) UROBILINOGEN, % ALBUMIN, Hct (Hematocrit), SMUDGE CELLS, NEUTROPHIL, and RBC DEFINITIVE. In some embodiments, more than 5 tests may be selected, for example, 6, 7, 8, 9, 10 or more tests in order to increase the accuracy of the prediction. In some embodiments, the probability to develop/having GDM may be determined based and the at least 5 tests results. High probability may be defined as e.g., higher than a threshold value, for example, 80%.

In some embodiments, a second method of predicting/detecting GDM in a woman may include: receiving the at least 5 of the following laboratory test results, obtained from the woman: NUCLEATED RBCS, HEMOGLOBIN A1C, D-DIMER, GLUCOSE, IRON, IRON BINDING, DEOXYCHOLIC ACID, CHENODEOXYCHOLIC ACID, CHOLIC ACID, TREPONEMAL AB TEXT, HIV 1 ANTIBODY, MYOGLOBIN_RANDOM URINE (QUANT), POC BICARBONATE, CHOLESTEROL_SERUM, TRIGLYCERIDES and VITAMIN D2_1_25. In some embodiments, more than 5 tests may be selected, for example, 6, 7, 8, 9, 10 or more tests in order to increase the accuracy of the prediction. In some embodiments, the probability to develop/have GDM may be determined based and the at least 5 tests results. High probability may be defined as e.g., higher than a threshold value, for example, 80%.

In some embodiments, a third method of predicting/detecting GDM in Jewish woman may include: receiving the at least 5 of the following laboratory test results, obtained from the woman: URINE KETONES and Magnesium Blood Level (Magnesium). In some embodiments, the probability to develop/have GDM in Jewish woman may be determined based and the tests results.

In some embodiments, a third method of predicting/detecting GDM in Jewish woman may include: receiving the at least 5 of the following laboratory test results, obtained from the woman: HEMOGLOBIN A1, ALK PHOSPHATASE, HEMOGLOBIN A2, Magnesium Blood Level and URINE PROTEIN. In some embodiments, the probability to develop/have GDM in Jewish woman may be determined based and the tests results.

In some embodiments, a similar method may be used for predicting/detecting of DGM in Muslim woman using the following tests: LEAD_BLOOD, HEMOGLOBIN A1C, MICROCYTES, CREATININE_FLUID, ACETONE and VITAMIN B12.

In some embodiments, a similar method may be used for predicting/detecting of DGM in Catholic woman using the following tests: HEMOGLOBIN A1, ALK PHOSPHATASE, HEMOGLOBIN A2, Magnesium Blood Level, URINE PROTEIN.

In some embodiments, a similar method may be used for predicting/detecting of DGM in Christian woman using the following tests: BLOOD UREA NITROGEN and ALT (Alanine Aminotransferase). Alternatively, the following tests may be used: ALT (Alanine Aminotransferase), AST, BLOOD UREA NITROGEN, URIC ACID, PLATELET COUNT, CSF ALBUMIN, GLUCOSE_RANDOM and CREATININE_FLUID.

In some embodiments, a first method of predicting/detecting preeclampsia in a woman may include: receiving the at least 5 of the following laboratory test results, obtained from the woman: ALK PHOSPHATASE, URINE PROTEIN, Magnesium Blood Level (Magnesium), URINE KETONES, URINE SUGAR, BLOOD UREA NITROGEN, CMV IGG AB, Diabetes Screen (GCT), HGB A2, NIL, MCHC, HGB F, HEMOGLOBIN A2 (QUANT), BAND NEUTROPHIL, O.D. VALUE, CSF ALBUMIN, LUPUS PTT, GLUCOSE_RANDOM, MUMPS IGM, 1 Hour (OGTT—One Instance), RBC, TOTAL PROTEIN, HERPES SIMPLEX 2 IGG, LEAD_BLOOD, TARGET CELLS, CHLORIDE, SICKLE CELLS, AST and CREATININE_FLUID. In some embodiments, the method may further include receiving the ethnicity of the woman. In some embodiments, more than 5 tests may be selected, for example, 6, 7, 8, 9, 10 or more tests in order to increase the accuracy of the prediction. In some embodiments, the probability to develop/have preeclampsia may be determined based and the at least 5 tests results. In some embodiments, the probability may further be determined based on the ethnicity. High probability may be defined as e.g., higher than a threshold value, for example, 80%.

In some embodiments, a second method of predicting/detecting preeclampsia in Jewish woman may include: receiving at least 4 the following laboratory test results, from tests taken by the woman: URINE PROTEIN, URINE SUGAR, MYOGLOBIN RANDOM URINE, NITROFURANTOIN, METAMYELOCYTE and PTT (Partial Thromboplastin Time). In some embodiments, the probability to develop/have preeclampsia may be determined based on the tests results

In some embodiments, a third method of predicting/detecting preeclampsia in a Christian woman may include: receiving at least 6 of the following laboratory test results, from tests obtained from the woman: LEAD_BLOOD, URINE UROBILINOGEN, HGB A2, HEMOGLOBIN S, URINE BENZODIAZEPINE, BASOPHIL. In some embodiments, the probability to develop/have preeclampsia may be determined based on the tests results.

In some embodiments, a fourth method of predicting/detecting preeclampsia in a Muslim woman may include: receiving at least 7 of the following laboratory test results, obtained from the woman: TEAR DROP CELL, GLUCOSE_RANDOM, B2 GLYCOPROTEIN IGM, LYMPHOCYTE, SICKLE CELLS, NEUTROPHIL and BILIRUBIN_TOTAL. In some embodiments, the probability to develop/have preeclampsia may be determined based on the tests results.

In some embodiments, a fifth method of predicting/detecting preeclampsia in a woman may include: receiving the at least 5 of the following laboratory test results, obtained from the woman: ANISOCYTOSIS, OVALOCYTES, EOSIN METAMYELOCYTE, ADP, RUBELLA_CONF.(EIA), CHOLESTEROL_SERUM, MCV (Mean Corpuscular Volume), DEOXYCHOLIC ACID, TRIGLYCERIDES, HIGH DENSITY LIPOPROTEIN, LOW DENSITY LIPOPROTEIN, MCH (Mean Corpuscular Hemoglobin), PERCENT FETAL CELLS, CHENODEOXYCHOLIC ACID, CHOLIC ACID, EST VOL FETAL BLOOD, CMV IGM AB, NRBC, URINE CANNABINOIDS, VANCOMYCIN TROUGH, PROGESTERONE, PROTEIN S ACTIVITY, TOTAL T3, FLUID RBCS, HCV RNA IU/ML, Hb Variant (Hb Variant—Maternal F), LIVER KIDNEY MICROSOM AB, CORD PCO2_ARTERIAL, FETAL HEMOGLOBIN, FLUID WBCS, GLUCOSE_FLUID, HEP B SURFACE AG, HEP C RNA LOG, TIMED URINE SODIUM, PROTEIN C, TREPONEMAL AB TEXT, PELGER-HUET, BETA-HCG QUANTITATIVE, MUMPS IGM, TREPONEMAL AB TEST, URINE BENZODIAZEPINE, METAMYELOCYTE, FREE INSPIRED OXYGEN, FLUID LYMPHOCYTES, LAMOTRIGINE, NIL, PARVO VIRUS INTERP, SUSCEPTIBILITY COMMENT and VZV TEXT. In some embodiments, more than 5 tests may be selected, for example, 6, 7, 8, 9, 10 or more tests in order to increase the accuracy of the prediction. In some embodiments, the probability to develop/have preeclampsia may be determined based and the at least 5 tests results.

In some embodiments, a first method of predicting preterm birth in a woman may include: receiving the at least 5 of the following laboratory test results, obtained from the woman: HEP B SURFACE AG, PROTEIN C, CORD PCO2_ARTERIAL, FLUID RBCS, HCV RNA IU/ML, LIVER KIDNEY MICROSOM AB, TIMED URINE SODIUM, WIDE RANGE CRP, Hb Variant (Hb Variant-Maternal F), PROGESTERONE, A/G, TOTAL T3, ACETYLCHOLINE RECEPTOR AB, AUTOFILING CONDITION, CPK, PROTEIN S ACTIVITY, RISTOCETIN, VANCOMYCIN TROUGH, HERPES SIMPLEX 2 IGG, NRBC, TOTAL T4, BLOOD HCG QUALITATIVE, FREE T3, HGB F, TOTAL BHCG 5TH, GLUCOSE, 3 Hour (OGTT—Three Instance), 3 Hour (OGTT—Three Instance), NUCLEATED RBCS, LIPASE, MYELOCYTE, OVALOCYTES, ANISOCYTOSIS, Maternal Plt (Platelets) and ATYPICAL LYMPHS. In some embodiments, more than 5 tests may be selected, for example, 6, 7, 8, 9, 10 or more tests in order to increase the accuracy of the prediction. In some embodiments, the probability to have the preterm birth may be determined based and the at least 5 tests results. High probability may be defined as e.g., higher than a threshold value, for example, 80%.

In some embodiments, a second method of predicting preterm birth in a Jewish woman may include: receiving the following laboratory test results, obtained from the woman: ANION GAP, B2 GLYCOPROTEIN IGM, TEAR DROP CELL/Dacrocyte, BLOOD UREA NITROGEN, Magnesium Blood Level (Magnesium) and PLATELET COUNT. In some embodiments, the probability to have the preterm birth in may be determined based and the tests results.

In some embodiments, a second method of predicting preterm birth in a Catholic woman may include: receiving the following laboratory test results, obtained from the woman: FERRITIN, DEOXYCHOLIC ACID, Na (Sodium), PELGER-HUET, ESBL, PLATELET COUNT. In some embodiments, the probability to have the preterm birth in may be determined based and the tests results.

In some embodiments, a second method of predicting preterm birth in a Muslim woman may include: receiving the following laboratory test results, obtained from the woman: TSH THIRD GENERATION, SUSCEPTIBILITY, TREPONEMAL AB (Anti Body), K (Potassium), Magnesium Blood Level (Magnesium), HEMOGLOBIN A2, URINE_GAMMA GLOBULIN, VITAMIN B12, MCV and NEUTROPHIL. In some embodiments, the probability to have the preterm birth in may be determined based and the tests results.

In some embodiments, a first method of predicting shoulder dystocia in a woman may include: receiving the at least 5 of the following laboratory test results, obtained from the woman: TREPONEMAL AB TEST, METANEPHRINES TOTAL, NEUTROPHIL, Hct (Hematocrit), LYMPHOCYTE, BiometryExcludeFL_v, HEMOGLOBIN S, AST, ANION GAP, FREE T4, BiometryExcludeAC_v, PT PATIENT, ELLIPTOCYTES, MONOCYTE, EOSIN METAMYELOCYTE, FLUID LYMPHOCYTES, MCHC, X (PPD Float—Two Instance), ALK PHOSPHATASE, O.D. VALUE, URINE UROBILINOGEN, WBC (Total WBC), BiometryExcludeBPD_v, SUSCEPTIBILITY COMMENT, Diabetes Screen (GCT), PLATELET COUNT, PTT (Partial Thromboplastin Time), HEMOGLOBIN C, NIL, URINE SUGAR, GAFetalWeigthLB_v, CSF ALBUMIN, BiometryExcludeHC_v, 1 Hour (OGTT—One Instance), HYALINE CAST-AUTO, URINE KETONES, TSH THIRD GENERATION, NRML_TEXT, URINE BENZODIAZEPINE, URINE PROTEIN, BASOPHIL, RBC DEFINITIVE, MICROCYTES, PARVO VIRUS INTERP, HEMOGLOBIN, HEMOGLOBIN A2, GLU POC RESULT, CMV IGG AB, URINE PH, CHLORIDE, HIV 1 ANTIBODY, AMYLASE, LUPUS PTT, TARGET CELLS, URINE SPEC GRAVITY and PELGER-HUET. In some embodiments, the method may further include receiving the race of the woman. In some embodiments, the method may further include receiving the spoken language of the woman. In some embodiments, the method may further include receiving the ethnicity of the woman. In some embodiments, more than 5 tests may be selected, for example, 6, 7, 8, 9, 10 or more tests in order to increase the accuracy of the prediction. In some embodiments, the probability to have the shoulder dystocia may be determined based and the at least 5 tests results. In some embodiments, the probability may further be calculated based on the race, spoken language and/or the ethnicity of the woman. High probability may be defined as e.g., higher than a threshold value, for example, 80%.

In some embodiments, a second method of predicting shoulder dystocia in a woman may include: receiving the at least 5 of the following laboratory test results, obtained from the woman: Maternal Plt (Platelets), ANISOCYTOSIS, OVALOCYTES, CREATININE_RANDOM URINE, MYELOCYTE, BODY TEMPERATURE, TOXOPLASMA IGM, BICARBONATE, PCO2, PH, PO2, AMP/SULBACTAM, BASE DEFICIT, NOVA SODIUM, LEAD_BLOOD, AZITHROMYCIN, HGB A, HGB A2, ANTITHROMBIN, URINE TP_RANDOM, LIPASE, ADP, PATIENTS HEIGHT_CM, RET %, Serratia marcescens, BiometryCER_v, TP PERIOD, NUCLEATED RBCS, TP VOLUME, VOLUME, TIMED URINE TOTAL PROTEIN, CALCIUM IONIZED and LACTIC ACID. In some embodiments, the method may further include receiving the race of the woman. In some embodiments, more than 5 tests may be selected, for example, 6, 7, 8, 9, 10 or more tests in order to increase the accuracy of the prediction. In some embodiments, the probability to have the shoulder dystocia may be determined based and the at least 5 tests results. High probability may be defined as e.g., higher than a threshold value, for example, 80%.

In some embodiments, a third method of predicting shoulder dystocia in a Jewish woman may include: receiving the following laboratory test results, obtained from the woman: URINE KETONES, URINE PROTEIN, METANEPHRINES TOTAL and ALT (Alanine Aminotransferase). In some embodiments, the probability to have the shoulder dystocia may be determined based on the tests results.

In some embodiments, a fourth method of predicting shoulder dystocia in a Jewish woman may include: receiving the following laboratory test results, obtained from the woman: ALT (Alanine Aminotransferase), URINE KETONES, METANEPHRINES TOTAL, HEMOGLOBIN VARIANT, MUMPS IGM, AST, GLUCOSE RANDOM, URINE PROTEIN, CO2, FREE INSPIRED OXYGEN, URINE PH, BAND NEUTROPHIL, O.D. VALUE, Na (Sodium), METAMYELOCYTE, RDW, LAMOTRIGINE, LUPUS PTT, NUM OF CELLS COUNTED, CHLORIDE, CALCIUM and URINE SUGAR. In some embodiments, the probability to have the shoulder dystocia may be determined based on the tests results.

In some embodiments, a fifth method of predicting shoulder dystocia in non-Hispanic or non-Latino woman may include: receiving the following laboratory test results, obtained from the woman: AST, 2 Hour (OGTT—Two Instance), FREE INSPIRED OXYGEN, URINE UROBILINOGEN and URINE KETONES. In some embodiments, the probability to have the shoulder dystocia may be determined based on the tests results.

In some embodiments, a sixth method of predicting shoulder dystocia in non-Hispanic or non-Latino woman may include: receiving the following laboratory test results, obtained from the woman: AST, URINE KETONES, NIL, PROTHROMBIN GENE, 2 Hour (OGTT—Two Instance), LAMOTRIGINE, URINE UROBILINOGEN, FREE INSPIRED OXYGEN, NUM OF CELLS COUNTED, MCHC, PTT (Partial Thromboplastin Time), ESBL, VITAMIN B12 and HEMOGLOBIN. In some embodiments, the probability to have the shoulder dystocia may be determined based on the tests results.

In nonlimiting example, the tests listed above for each method may be selected according to the corresponding level of importance, for example, according to the order at which the tests are listed herein above.

In some embodiments, the listed laboratory tests may be conducted using any laboratory method known in the art and the invention is not limited to any specific laboratory method.

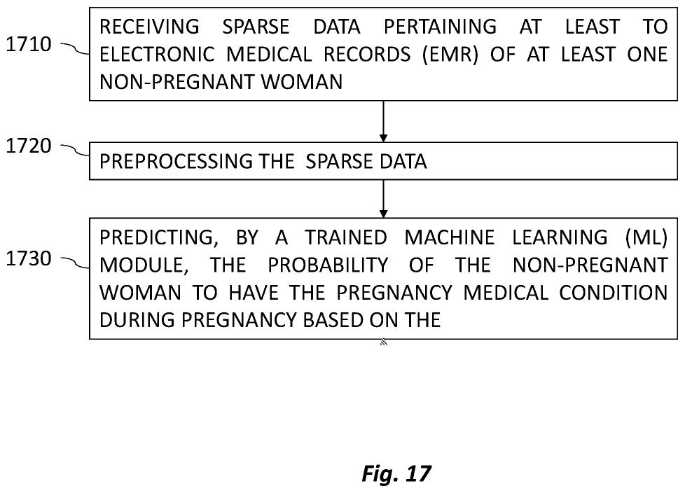

In some embodiments, the method may further include training a machine learning (ML) module to predict the medical condition based on the completed data and the level of importance. In some embodiments, the processor may train ML module 50 to predict the medical condition in a patient not yet having any signs or symptoms of the medical condition. For example, ML module 50 may be trained to predict pregnancy medical condition in non-pregnant woman, as discussed with respect to the method of FIG. 17. In some embodiments, ML module 50 may be trained to predict developing Hypothyroidism at adulthood in teenagers.

In some embodiments, the method may further include predicting, for the at least one patient, by the trained ML module, a future appearance of the medical condition based on the completed data, which following the above steps, methods and modules may not consider to be a sparse data. In some embodiments, the processor may receive an EMR of a specific patient and/or medical tests and personal data of the specific patient. In some embodiments, the processor may use the trained ML module 50 to predict a future appearance of the medical condition, for example, precondition diagnosis 50A. For example, system 100 may use any one of its modules, using any one of the methods disclosed herein to predict the probability of a teenager to develop PCO at adulthood.

In some embodiments, the method may include providing a treatment recommendation, for example, treatment recommendation 50B. System 100 may use any mathematical and/or statistical model, ML and the like to provide the treatment recommendation.

In a first nonlimiting example, a treatment recommendation for GDM or other types of diabetes may be determined by system 100 by matching between physician profile to patient profile based on a “successful”/“Fail” result”, as disclosed herein above. System 100 may predict what will be the result in a specific decision made the physician on a specific patient on any other action/recommendation/treatment. The system may calculate the various possibilities for approved protocols, and label them as “the policy”. The policy may be defining the “rules” in the game. The system may play a “chess” like game in order to see which kind of recommendation will be the most beneficial having a specific patient profile. In such case the patient's and physician's profiles are called “State 0”/“environment 0”

In some embodiments, different recommendations/actions taken by the physician may be used. The system may compare between the actual results, labeled with “success”/“failure” and based on that may reward the model. The rewards ranges may be between −1 to 1. In every state/environment that the system may calculate the reward based on the distance from the expected result. The system may used one method for the final result, like condition—0—prediction was that the patient is healthy, 1—if the patient was diagnosed with the condition.

In a second nonlimiting example, the system may consider the results in breakpoints. For example, the system may identify the changes in the glucose levels, having a specific patient profile and a various recommendation. The stage of completion missing elements may help to make more times these breakpoints and to have more accurate results, as shown in equation (9).

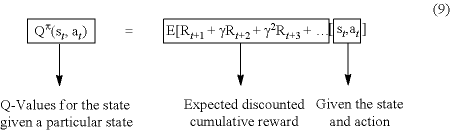

In a third nonlimiting example, a combination between the first and second nonlimiting examples for treatment recommendation may be conducted by the system. System 100 may change different elements in the patient profile and may try to run equation (9) again to see what the changes are, as illustrated in FIG. 5B. Based on that process the system may create behavioral equations of each parameter. In some embodiments, the delta of change per parameter may enable to better understand the impact of the change on other parameters and on the final result. By doing so, system 100 may also defined the causality for each condition based on various profiles and effects.

In some embodiments, the method may include reinforcement between prediction and detection, in order to reduce misdiagnosis. In some embodiments, system 100 may feed the predicted conditions back to the system to further train system 100 to enrich the data and/or to optimize the performances and/or to update the predictions. As illustrated in FIG. 5C, following the processing and analyzing of various parameters 560, the predicted conditions 570 may be fed back to system 100 at 580. In some embodiments, predicted conditions 570 may be given a lower weight in comparison to the conditions that may already be diagnosed. The system may further receive confirmations if a predicted condition was eventually diagnosed and may assign higher a weight to the confirmed prediction in the further training of system 100 (e.g., training ML module 50).

Some specific examples related to the prediction of GDM in women and correlation between different parameters are given in FIGS. 7-16. As should be understood by one skilled in the art, the flowing results and tables are given as examples only, and the invention as a whole is not limited to predicting GDM. The same methods, modules, modalities and processes can be used for predicting other medical conditions.

FIGS. 7A and 7B are graphs showing the relationships between blood pressure (BP) (systolic and diastolic respectively) prior to pregnancy and the probability to develop gestational diabetes according to some embodiments of the invention. As shown in the graphs the relations between any given BP prior to pregnancy and developing GDM are not direct (e.g., linear). For instance, in cases that diastolic levels are around 55 mmHg-65 mmHg the probability to develop GDM decreases, but when the diastolic pressure is going out from this range in any direction, the probability to develop GDM increases.

Reference is now made to the table in FIG. 8 that shows the correlation between vital signs tests prior to pregnancy and developing GDM according to some embodiments of the invention.

As shown in FIG. 8 some vital signs, such as, intravascular diastolic BP, intravascular systolic BP, body weight and heartbeat were more relevant to DGM other than other. For example, the measure body weight are more related, thus higher importance in predicting GDM than BMI. Therefore, the processor may give an importance score (e.g., a value) to each vital sign, as shown in the table of FIG. 9. In some embodiments, the score/enumeration may be given using reliability module 416.

FIGS. 10-15 shows the influence of demographic parameters prior the commencement of pregnancy on the probability of developing a GDM during the pregnancy

In some embodiments, the processor may conduct specific calculation of the probability to develop GDM per parameter as shown in FIG. 10-14. The processor may classify independent demographic variables to (GDM/Non GDM) using of a hybrid algorithm (e.g., the hybrid methods). The processor may find the dependency of the demographic variables in the classification. The table of FIG. 10 shows the probability and relations between religion prior to pregnancy and developing GDM.

The table of FIG. 11 shows the probability and relations between race prior to pregnancy and developing GDM. As shown that both black and white Hispanic women have the highest probability of developing GDM.

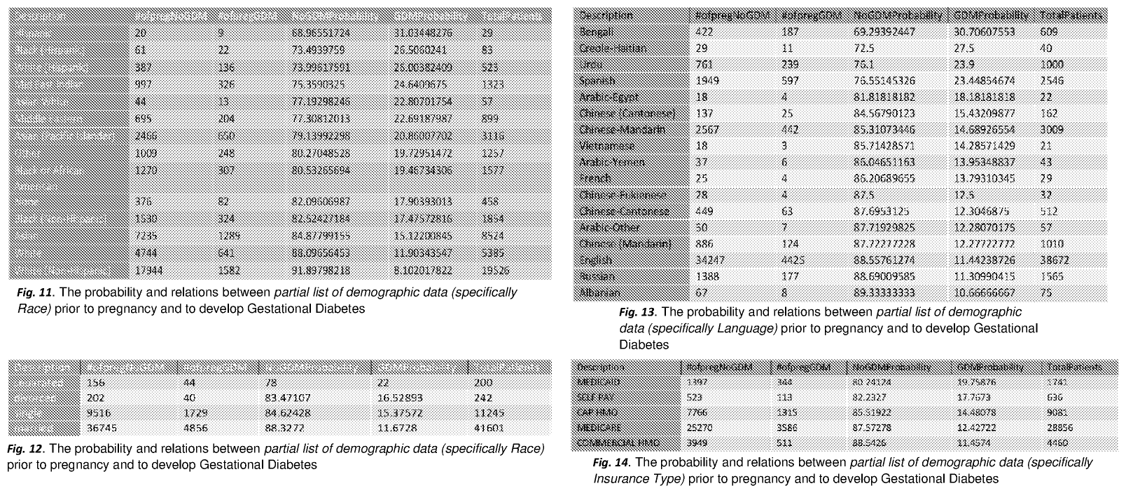

The table of FIG. 12 shows the probability and relations between marital status prior to pregnancy and developing GDM. As shown separated women have the heist probability of developing GDM.

The table of FIG. 13 shows the probability and relations between spoken language prior to pregnancy and developing GDM. As shown Bengali speaking women have the highest probability of developing GDM.

The table of FIG. 14 shows the probability and relations between medical insurance type prior to pregnancy and developing GDM. As shown women having MedicAid insurance (in USA) have the highest probability of developing GDM.

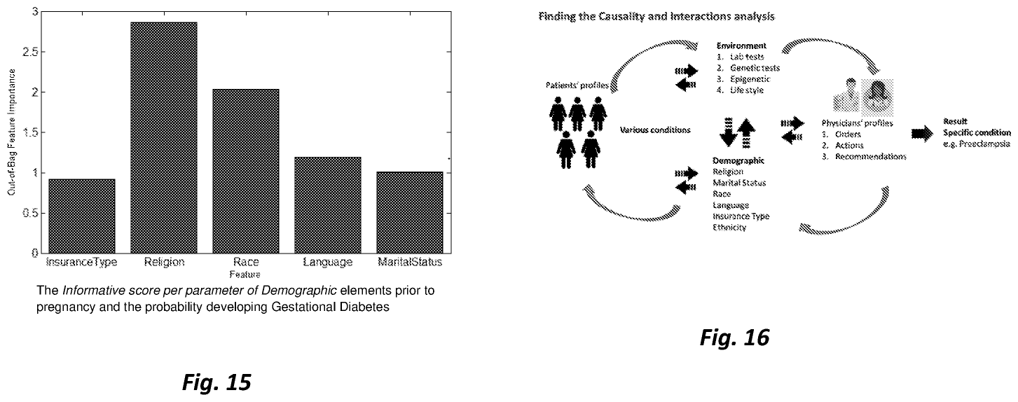

In some embodiments, the processor (e.g., controller 2) may assign a score to the demographic parameters. FIG. 15 is a graph showing informative score (e.g., out of bag feature importance) given to demographic parameter prior to pregnancy based on the probability developing GDM. As can clearly show in the graph the most influential parameter is the religions, which is almost 50% more influential than the second most influential parameter-race.

In some embodiments, system 100 may combine and set relations between parameters from different areas. In some embodiments, the correlation (linear relation) and the informative score (nonlinear relations) of a combination of parameters were examined. In some embodiments, the question “is there any relation between a combination of parameters and the probability to develop GDM?” was answered. A well-known example is the BMI which is the combination between the height and weight that creates a new and more informative variable, since to separately look at the weight and height will not be sufficient.

In some embodiments, parameters such as number of pregnancies, the number of times that the patient had GDM in the past pregnancies and the number of pregnancies that the patient had GDM before the current emergency

# of pregnancy

# of times that the patient had GDM in the past pregnancies

A binary question that represents if the patient had GDM in the previous pregnancy, for example, have the patient had a GDM in the previous pregnancy? (0/1)

In some embodiments, a complex systems approach may be used. System 100 may include a hierarchal data method that ordered as follow: such an order is illustrated in FIG. 4B. The highest reliability score is given to Admission, Discharge, Transfer (ADT) data received form hospitals. The second highest reliability score is given to laboratory tests, vital signs, demographic data, etc. The third highest reliability score is given to physicians' orders message (ORM) for example, may involve changes to an order such as new orders, cancellations, information updates, discontinuation, etc. The fourth reliability score is given to physician's orders. The fifth highest reliability score is given to medications that were given to the patient. The sixes reliability score is given to observation result (OUR), e.g., allergies, procedures, etc. The seventh reliability scores given to Detailed Financial Transactions (DFT)

In some embodiments, system 100 may analyzed each data type separately, and then explored the relation between the data types. For example, it was found that there is a relation between abnormal results from laboratory tests of Thyroid-Stimulating Hormone (TSH), tests performed on the ultrasound unit, a history of early macrosomic infant or Hx of baby weighing more than 4500 gm or 9 lbs. 8 oz. and GDM.

The above nonlimiting method was applied for a specific patient that was sent to conduct laboratory tests and was found to be diagnosed with a thyroid disease. When checking the hypostasis that there is a relation between thyroid functioning and developing GDM, it was found that the patient was also diagnosed with GDM two days form the diagnosis of the thyroid disease. In some embodiments, it was found that 26% of the women who diagnosed with GDM, developed thyroid disease, and vice versa. These complex relations occur always between different systems in the human body.

Reference is now made to FIG. 16 which illustrates the general flow of information and data received from the EMR and other information sources related to woman (given as a nonlimiting example only) diagnosed or to be diagnosed with various conditions. The system may include environmental information for example, laboratory tests, genetic tests, epigenetic data and lifestyle information. The system may include demographic information, for example, religious, race, marital status, ethnicity, insurance type and language. The system may include a physician profile, for example, the physician's recommending, orders, and diagnoses. The outcome of the system may include a diagnosis of a specific condition, for example, preeclampsia.

Reference is now made to FIG. 17 which is a flowchart of a method of predicting, by at least one processor or controller (e.g., element 2 of FIG. 1A), a pregnancy-related medical condition in a non-pregnant woman or determining the pregnancy-related medical condition in a pregnant woman. A pregnancy-related medical condition, according to some embodiments of the invention may include any abnormal condition that may occur during pregnancy, for example, GDM, preeclampsia, preterm, hypothyroidism, hyperthyroidism, cardiovascular diseases, postpartum hemorrhage, fetal heart disorder, fetal growth disorder, intra-uterine growth restriction (IUGR) and the like. The method of FIG. 17 may be conducted by system 100 using one or more computing devices (e.g., element 10 of FIG. 1A).

In step 1710, processor 2 may receive sparse medical data of a woman. In some embodiments, step 1710 may be substantially the same as step 510 of the method of FIG. 5.

In step 1720, the processor may preprocess the sparse medical data. In some embodiments, step 1720 may be substantially similar to step 520 of FIG. 5. In some embodiments, the method may further include completing the sparse data by adding at least a portion of missing data using a cross-validation process, as disclosed herein (e.g., with respect to step 530 of FIG. 5).

In step 1730, the processor may predict, by a trained ML module (e.g., element 50 of FIG. 1B), a probability of woman to have the pregnancy-related medical condition during pregnancy (e.g., during a future pregnancy). In some embodiments, the inclusion/exclusion criteria for training ML 50, may be related to pregnant women diagnosed with the medical condition. An example for the implementation of such criteria is given in table 1 herein above. In some embodiments, the exclusion criterion may include excluding parameters included in the medical data that were received during any of the woman previous pregnancies.

Reference is now made to FIG. 18 which is a flowchart of a method of determining a physician profile according to some embodiments of the invention. The physician profile may allow to predict what will be a decision/diagnosis made by a physician thus may asses the reliability of the physician's decisions/diagnoses. The method of FIG. 18 may be conducted by system 100 using one or more computing devices 10. In step 1810, the processor may receive a plurality of decisions made by the physician in response to a plurality of medical conditions, from EMR. In some embodiments, the physician profile may include a lookup table connecting, parameters (e.g., tests, symptoms and the like), diagnosis and patient profiles.

In step 1820, the processor may determine a plurality of patients' profiles, based on data received from the EMR. The processor may conduct any required step disclosed in steps 510-520 of FIG. 5 in order to preprocess and complete the sparse data (either related to the patient or the physician) received from the EMR. In step 1830, the processor may associate each decision with a patient's profile. For example, for all women diagnosed, by a specific gynecologist with preeclampsia, the processor may group the women's profiles into a preeclampsia related group.

In step 1840, the processor may determine the physician profile based on the association. In some embodiments, the processor may cluster sub-group of patients from plurality of patients based on similar decisions made by the physician for a given medical condition. For example, the processor may find a group of women having similar profiles (e.g., as set forth by use similarity module 411) all diagnosed with preeclampsia by the same gynecologist, accordingly, the processor may determine that for a woman having high similarity to the group showing the same symptoms, it is very likely that the specific gynecologist will diagnose that woman also with preeclampsia. In some embodiments, determining the physician profile may include determining, for a given medical condition and a patient's profile a predicted decision.

In some embodiments, processor 2 may assess whether the diagnosis was correct by, for example, comparing the diagnosis of a specific physician to diagnosis given to similar women (e.g., as set forth by use similarity module 411) by one or more other physicians. In some embodiments, the processor 2 may determine whether the diagnosis could have been given without the intervention of the physician, for example, based on laboratory tests only. In some embodiments, the processor 2 may replace the physician and diagnose the women instead of the physician. In some embodiments, the processor 2 may give a reliability score to each doctor and may determine how much weight to give the decisions and diagnoses of each specific physician in relation to the training of ML module 50.

In some embodiments, the processor 2 may determine for each physician profile a decision diversity function in identical medical situations. The decision diversity function may allow the processor to correct the physician decision in identical medical situation. In some embodiments, the processor 2 may use physician profile module 415 to correct parameters related to physician inputs based on the determined decision diversity function. In some embodiments, physician profile module 415 may use mathematical and/or statistical methods to create the decision diversity function. The module may use any mathematical function for determining the decision diversity function.

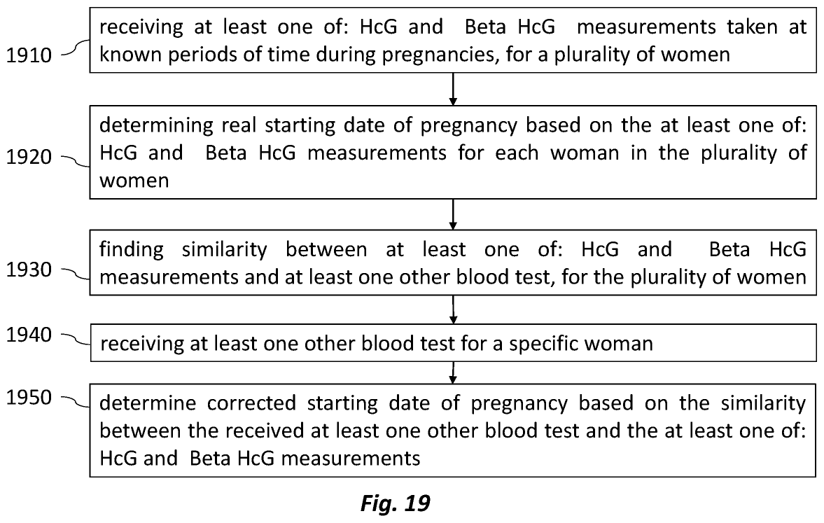

Reference is now made to FIG. 19 which is a flowchart of a method of determining, by at least one processor, a starting date of pregnancy according to some embodiments of the invention. The method of FIG. 19 may be conducted by system 100 using one or more computing devices (e.g., element 10 of FIG. 1A). An illustration of the method is shown in FIG. 20.

In step 1910, the processor may receive at least one of: HcG and Beta HcG measurements taken at known periods of time during pregnancies, for a plurality of women. In some embodiments, the at least one of: HcG and Beta HcG measurements may be received from the EMR of the plurality of women.

In step 1920, the processor 2 may determine a real starting dates of pregnancy (that may be different than the date of LMP) based on the at least one of: HcG and Beta HcG measurements for each woman in the plurality of women. As is known in the art the at least one of: HcG and Beta HcG measurements is one of the most accurate measurements for determining starting dates of pregnancy, as the level of at least one of: HcG and Beta HcG at each stage/date of the pregnancy is known. Accordingly, knowing the date of at least one of: HcG and Beta HcG test and the level of the measure at least one of: HcG and Beta HcG may allow to accurately calculate the real starting dates of the pregnancy.

In step 1930, the processor may find similarity between at least one of: HcG and Beta HcG measurements and at least one other blood test, for the plurality of women. For example, the processor 2 may use parameter similarity module 412 to find similarities between at least one of: HcG and Beta HcG measurements and tetraiodothyronine—T4, as illustrated in FIG. 20. The processor 2 may find similarity (e.g., linear or non-linear correlation) between the levels of HcG and/or Beta HcG and the levels of the at least one other blood test at similar stages of the pregnancy.

In step 1940, the processor may receive the at least one other blood test for a specific woman. For example, the processor may find in the woman's EMR a date and level of tetraiodothyronine complement C4, T cells, WBC, and the like conducted after the conceiving.

In step 1950, the processor 2 may determine a corrected starting date of pregnancy for the specific woman based on the similarity between the received at least one other blood test and the at least one of: HcG and Beta HcG measurements. Therefore, a method according to embodiments of the invention may allow to use other blood test as an indicator for determining a corrected starting date of pregnancy.

The above disclosed methods and systems can be used for additional applications, for example, in assisting medical services providers, such as, medical insurance companies, hospitals, pharmaceutical companies and the like to be much more efficient in providing their services.

The above disclosed methods and systems can be used for additional applications, for example, in assisting medical services providers, such as, medical insurance companies, hospitals, pharmaceutical companies and the like to be much more efficient in providing their services.

In some embodiments, medical insurance companies may utilize embodiments of the disclosed methods and systems for the following applications.

In some embodiments, medical insurance companies may use the above disclosed methods and systems to conduct precise paramedical test. The medical insurance companies may create a precise list of paramedical tests based on the required and/or necessary medical tests per condition based on a specific profile. This process is not only cost effective, but also has an impact on the medical field.

In some embodiments, medical insurance companies may use the above disclosed methods and systems to conduct risk modeling based on precise patient profile. In some embodiments, the methods described herein above may use to predict the probability of a patient to develop the condition, even if the patient is not aware of the condition. By doing so, the medical insurance companies may create a risk modeling profile that contains all the current risks and future risk per patient.

In some embodiments, the method may include determining a risk level for each patient. In some embodiments, system 100 may receive a set of important medical conditions and may determine the probability of a specific patient to develop each one of the medical conditions in the set, for example, using trained ML module 50. In some embodiments, the risk level may be determined based on the plurality of determined probabilities, for example, by, summing, multiplying or conducting any mathematical method using the determined probabilities.

In some embodiments, medical insurance companies may use the above disclosed methods and systems to conduct an automated and precise prior authorization. In some embodiments, the use of the methods that were mentioned above may enable to determine whether a treatment will be effective on a specific patient or not and will enable to recommend the insurance company to approve it or not. For example, system 100 may use physician profile module 415 for evaluating decisions that may have been made by a doctor. The system may determine whether the decision is correct or whether it is the most accurate for the specific patient. The system may provide an alternative approach, using for example, any one of the treatment recommendation methods disclosed herein above.

In some embodiments, medical institutes, such as hospitals, ministries of health and the like, may use systems and methods for financial and/or budget planning (e.g., expected expenses). In some embodiments, system 100 may obtain predicted conditions for one or more patients, and used the obtained predictions to calculate or determine a budget that may be required to deal with the predicted conditions. For example, in medical institutes, specific medical conditions may have or may be attributed corresponding financial codes (e.g., ICD-10 in the US). Each of the codes may be related to a specific budget, that may be known (e.g., by the medical institute) to cover the costs of dealing with the corresponding medical condition. Therefore, aggregating the occurrence of predicted conditions may allow medical institutes to evaluate their combined costs. Furthermore, collecting all the predicted conditions may allow to evaluate the required resources for dealing with the conditions, for example, laboratory tests, medical imagery tests, medical staff, hospital beds, medications and the like.

In some embodiments, the evaluation of the financial aspects may be done to a single patient, for example, for determining their appropriate insurance rates. In some embodiments, the evaluation of the financial aspects may be done to groups of patients.

For example, the ministry of health in Canada may conduct a full survey using all the EMR of the entire population of Canada, in order to predict what are the expected conditions (e.g., illnesses) in the general population in the upcoming year, so as to calculate at least a portion of the ministry's budget. In another example, an insurance company may conduct a full survey on the entire population of women (covered by the insurance company) to predict gynecological conditions, and to evaluate the gynecological budget of the company. In yet another example, hospitals may use system 100 to evaluate the number of beds, plan the medical staff, determine the amount of required laboratory equipment and tests, determine the amount of required medical imagery equipment, etc., so as to deal with the expected or predicted medical conditions.

In some embodiments, pharmaceutical companies may utilize the disclosed methods and systems for the following applications.

In some embodiments, pharmaceutical companies may use the above disclosed methods and systems to identify and enroll of patients for clinical trials. In some embodiments, the system may to identify patients who has high likelihood to develop any specific condition and to enroll these patients to the clinical trials and to shorten the period of clinical trials.

In some embodiments, pharmaceutical companies may use the above disclosed methods and systems to personalize medicine. In some embodiments, at least some of the modules disclosed herein above may allow to use the precise patient-diagnosis profiles as an open window for determining novel and personalized medications list per group of patients and/or per specific patient, for example, using any one of the treatment recommendation methods disclosed herein above. In some embodiments of the invention the system may allow to discover new biomarkers/genetic tests to identify medical conditions, using for example, the demographic parameters disclosed herein above.

Embodiments of the invention may provide a practical application for predicting or evaluating a medical condition in a patient. Embodiments of the invention may include an improvement over currently available technology of diagnostics tools (e.g., using ML-based methodology to predict a medical condition) by performing said prediction based on highly sparse data (e.g., originating from existing medical records). As explained herein, a system and method according to embodiments of the invention may allow to use highly sparse medical data in order to determine a variety of required medical and non-medical related information.

Embodiments of the invention may process the sparse data in order to provide the processed sparse data to the ML module in the most complete way that may allow more efficient use in the ML module and receiving more reliable prediction from the ML module.

Embodiments of the system may perform a bidirectional process, including: (a) starting from the final diagnosis of a medical condition going backwards, step by step finding the required parameters for predicting the medical condition; and (b) starting from the required parameters to find the probability of having the medical condition.

Embodiments of the system may include a practical improvement over currently available technology by using said sparse data to set the required tests and parameters for diagnosing the medical condition for a specific patient or for a group of patients. The system can determine a set of necessary medical tests and recommend the doctors to send the patient or group of patients to take the medical tests.

Embodiments of the system may allow medical service providers, such as, medical insurance companies, hospitals, pharmaceutical companies and the like to be much more efficient in providing their services.

The development process and algorithms may involve many steps. A unique patient profile may be created by each data point receiving a weight, which may be based on a reliability calculator. Next, each of the parameters received an importance score that may be calculated precisely based on novel mathematical models. Using this method, the importance for each parameter may be found in the decision of developing GDM or other medical conditions, and also checked the various degrees of the relationship between the parameters and the probability of developing GDM or other medical conditions. The probability, causality and the reliability results were then combined to provide a result for developing GDM or other medical conditions.

The inventor has found that the ability to use novel mathematical algorithms to identify important elements for prediction of a disease, which otherwise would not be identified. The inventor found that there were hundreds of parameters that had different importance, likelihoods, relation and interaction between parameters and the probability of developing gestational diabetes. In some embodiments, retrospectively, a system and method according to the invention was able to detect differences between the parameters and ranked their performance to be able to predict at preconception and early gestation the risk of developing GDM and other conditions As should be understood by one skilled in the art, described herein only a few examples, as the full data training of this algorithm is beyond the scope of this disclosure.

Currently, in the medical field, only models have been used for prediction of disease. A system and method according to embodiments of the invention may use an artificial intelligence that uses algorithms. The main difference between models and algorithms is that an algorithm is a mathematical technique derived by statisticians and mathematicians for a particular task, e.g. prediction, while model is a set of strict rules to follow. In addition, the system and method according to embodiments of the invention may have the capability of pulling and analyzing data not only from structured and discreet data elements, but also from narrative and free text data elements.

Accordingly, system and method according to embodiments of the invention is a promising solution that may decrease unnecessary medical tests that can decrease the number of complications, and in turn decrease healthcare costs related to unnecessary medical tests. Furthermore, precise early detection of disease may allow opportunities for early interventions that in turn will decrease morbidity. Other adverse outcomes should be trained under this model. In addition, system and method according to embodiments of the invention may predict GDM or other pregnancy conditions at preconception and early pregnancy in order to enable early intervention such as diet and life style modifications.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention. Further, features or elements of different embodiments may be used with or combined with other embodiments.

1. A method of selecting, by at least one processor, required parameters for prediction or detection of a medical condition, comprising:
receiving sparse data pertaining at least to electronic medical records (EMR) of at least one patient;
preprocessing the sparse data;
completing the sparse data by adding at least a portion of a missing data using a cross-validation process; and
selecting the required parameters from the completed data.

2. The method of claim 1, wherein selecting the required parameters is by a module adapted to:
receiving sparse data pertaining to at least EMR of a plurality of patients; preprocessing the sparse data;
completing the sparse data, pertaining to EMR of a plurality of patients, by adding at least a portion of a missing data using the cross-validation process; and
arranging the parameters in the completed data according to their level of importance.

3. (canceled)

4. The method of claim 2, wherein the level of importance is determined by information gain calculations comprising:
receiving for each parameter in the sparse data:
a first number of patients included in a first group of patients to which the EMR data comprises the parameter; and
a second number of patients included in a second group of patients diagnosed with the condition, the second group is selected from the first group.

5. The method of claim 1, further comprising:
identifying in the required parameters, one or more parameters related to medical tests; and
determining a list of required medical tests for prediction or detection of a medical condition based on the identified parameters.

6. The method of claim 1, wherein receiving the sparse data pertaining to the EMR is for a group of patients belonging to at least one category; and
wherein the required parameters are selected to best fit the group of patients.

7. The method of claim 1, further comprising: training a machine learning (ML) module to predict or detect the medical condition based on the completed data and the level of importance.

8. The method of claim 7, further comprising:
predicting or detecting, for the at least one patient, by the trained ML module, a future appearance of the medical condition based on the completed data.

9. The method of claim 7, further comprising:
receiving a set of medical conditions;
predicting for the at the least one patient a probability for future appearance of each medical condition in the set; and
determining a risk level for the at the least one patient based on the predicted probability of each medical condition in the set.

10. The method of claim 1, wherein completing the sparse data comprises: completing parameters missing from the sparse data with parameters having a sufficient similarity, and wherein the similarity is determined based on at least one of:
a similarity between patients, similarity between parameters and a combination thereof.

11. The method of claim 10, further comprising:
determining a first reliability level of each parameter based on a time associated with the parameter.

12. The method of claim 10, further comprising:
determining a second reliability level of each parameter based on number of occurrences of each parameter for different patients.

13. The method of claim 1, further comprising: determining a physician profile for a plurality of physicians;
determining for each physician profile a decision diversity function in identical medical situation; and
correcting parameters related to physician inputs based on the determined decision diversity function.

14. (canceled)

15. The method of claim 2, wherein the preprocessing of the sparse data comprises:
inclusion of data received only from patients diagnosed with the medical condition; and
inclusion of the data received from patient in a precondition.

16. (canceled)

17. The method of claim 1, wherein at least one parameter from the sparse data is a monitored parameter, and the method further comprising:
detecting at least one abnormality in the monitored parameter; and assigning a representing value to the detected at least one abnormality.

18. The method of claim 1, wherein the preprocessing of the sparse data comprises:
identifying category dependent parameters in the sparse data; and assigning a score for each category.

19. The method of claim 18, wherein the at least one category is selected from: religion, ethnicity, race, spoken language, age, gender, citizenship, place of birth, and place of living, social economic level, insurance type, education, occupation.

20. The method of claim 19, wherein the at least one category dependent parameter is one of: a genetic parameter and epigenetic parameter.

21. The method of claim 18, wherein the at least one category dependent parameter is a geographic parameter related to a location of the patient selected from: radiation levels at the location, temperatures at the location, humidity levels at the location and altitude of the location.

22.-32. (canceled)

33. The method of claim 1, wherein the preprocessing of the sparse data comprises: normalizing time dependent parameters in the sparse data received from different patients to a single timeline.

34. The method of claim 33, further comprising:
dividing the timeline into time intervals;
associating each time dependent parameter with a specific time interval; determining a decay rate parameter for each time interval; and
calculating a weight of each time dependent parameter using the corresponding decay rate parameter.
